(19) United States
(12) Patent Application Publication
(10) Pub. No.: US 2024/0412823 A1
(43) Pub. Date: Dec. 12, 2024

(54) VQE FOR IMPROVED DFT SIMULATIONS

(71) Applicant: Phasecraft Limited, London (GB)

(72) Inventors: Toby CUBITT, London (GB); Evan SHERIDAN, London (GB); Raul Antonio Santos SANHUEZA, London (GB)

(21) Appl. No.: 18/733,534

(22) Filed: Jun. 4, 2024

(30) Foreign Application Priority Data
Jun. 12, 2023 (GB) 2308768.7

(51) Int. Cl. G16C 10/00; G16C 60/00

(52) U.S. Cl. CPC G16C 10/00; G16C 60/00

(57) ABSTRACT

A method of performing Kohn-Sham Density Functional Theory, DFT, calculations, for simulation of a material, chemical, or biological system includes: (i) constructing a model Hamiltonian of the system; (ii) inputting an electron density; (iii) constructing a Kohn-Sham potential and (iv) executing a quantum Kohn-Sham DFT algorithm iteration using the electron density and the Kohn-Sham potential to compute an updated electron density and an updated total energy. The Kohn-Sham potential is constructed by: (1) providing a second Hamiltonian based on the model Hamiltonian; (2) calculating a ground state energy of the second Hamiltonian, as a function of electron density; (3) computing a quantum-obtained exchange correlation energy functional, as a function of electron density; (4) obtaining a quantum-obtained exchange correlation potential functional by differentiation of the quantum-obtained exchange correlation energy functional; and (5) using the potential functional from (4) to construct the Kohn-Sham potential.

### FIELD OF THE INVENTION

The present application relates to methods and apparatuses for performing hybrid quantum-classical density functional theory calculations. In particular, the application relates to methods for improved DFT simulation of material, chemical, and biological systems on noisy intermediate scale quantum processors.

### BACKGROUND OF THE INVENTION

Classical DFT is the principal and most widely used method to simulate a broad range of ground state properties for materials systems at a quantum mechanical level. Understanding and describing the properties materials systems is a notoriously difficult problem as these constitute large, interacting, and inhomogeneous many-body problems that warrant quantum simulation for a precise understanding of their dynamics. The central quantum mechanical quantity that encapsulates these processes is the many body wavefunction, which scales exponentially with the number of particles. As a result, the purview of exact simulation of materials on classical computers is restricted to small system sizes, and a number of well-reasoned approximations across different time and length scales are required to make consistently reliable predictions. One of the main motivations of DFT is to avoid the classical intractabilities of simulating many body systems, and its many successes have bolstered its popularity in many areas of physics and chemistry. In the most common formulation of DFT, its algorithmic complexity scales as O(N³), with N being the number of particles, whereas the full quantum many body simulation scales as O(4^{N}).

At the atomic scale, ab initio Density Functional Theory (DFT) has the premier status among computational methods that compute ground state electronic and structural properties for a broad spectrum of atoms, molecules and materials. The essence of the approximation is to replace the many body wavefunction as the central quantity that is solved for—with that of the one body density—which instead is only a function of the 3 spatial dimensions. Thus, the exponential classical computational cost of computing the ground state of an interacting many-body wave system is replaced with a polynomial time classical variational algorithm for a fictitious non-interacting single particle system. Under certain conditions the solution of this non-interacting system is guaranteed to produce the ground state properties of the interacting system—allowing for tractable and efficient simulations beyond the scope of exact classical wavefunction methods. This is achieved by absorbing all of the many-body complexity into something known as the exchange correlation (XC) functional; however this is generally QMA-hard to compute.

Perhaps the best-known implementation of DFT is its ab initio formulation for the many electron Hamiltonian within the Born-Oppenheimer approximation, where the ions generate a static, periodic varying external potential. In Kohn-Sham DFT, the system is then mapped from the many-particle Hilbert space onto the effective non-interacting Kohn-Sham space, via the Kohn Sham potential. Particularly well-known parametrisations of the XC potential, which is the only unknown contribution within KS potential, involve exact computations of the homogeneous electron gas.

DFT is intrinsically limited by the XC functional design and choice, where simple and compact approximations have shown to be the most useful, interpretable, and computationally efficient. Classical DFT replaces the exact XC functional with various approximations, establishing the field of approximate functional design, which has enjoyed abundant success for non-interacting systems. The struggle of DFT to fully express the many body correlations present in the ground state of interacting electronic systems fundamentally limits the present applicability of DFT. In principle, the exact functional is only accessible through a full many body solution, which cannot be achieved on classical computers except in a very small number of scenarios. Therefore, the potential for success of DFT calculations for simulations to inform practical applications implicitly relies on compact approximations of the unknown exchange correlational functional, and so the design of such functionals has become a primary pillar of classical materials simulation research. Thus, we have seen hundreds of approximate XC energy functionals proposed over more than 50 years, for both continuous basis sets as well as model Hamiltonians. These exchange correlation functionals are used in virtually every facet of atomistic materials modelling. This has resulted in the foundational papers of DFT becoming some of the most cited works in the scientific literature and the birth of the field of ab initio materials design. Although DFT is a very successful method for studying weakly correlated electronic systems, a number of challenges still remain, from choosing the most appropriate functional for the system at hand to the general failure of XC functionals when applied to strongly correlated systems.

When applying classical functionals to strongly correlated electronic systems, DFT can often qualitatively fail in its predictions. There are two prevalent DFT camps that separate the fields of weakly and strongly correlated electronic systems. Typically, real space DFT is used to study weakly correlated systems, while lattice Hamiltonians are used for the latter, to fully account for all of the electronic correlations present in strongly correlated system.

The lattice formulation of DFT for model Hamiltonians is an active area of research, where minimal models, such as the Hubbard model or tight binding Hamiltonians, are fully characterised as a Kohn-Sham system. In this formalism, coined Lattice DFT (LDFT),density functionals are constructed for lattice models, which can then be used in iterative Kohn-Sham DFT algorithms.

XC functional design for correlated lattice models has also been explored classically in the context of various lattice inhomogeneities. It was first used in semiconductor models but has since been more routinely applied to one dimension Hubbard chains where exact parametrisations of the XC functional are available, based on the Bethe Ansatz. The role of the discontinuity in XC potentials on model and real systems is particularly important. In particular, implementing the Bethe Ansatz LDA functional for the one dimensional Hubbard model has allowed comparison to investigate how the XC discontinuity relates to molecular (hydrogen chains, dimolecules) and periodic materials systems. Exact functional results have been benchmarked against a number of state of the art classical functionals.

Exact Diagonalisation can be used to compute an approximation to the exchange correlation functional. In 2d this approach has been used to highlight ground state properties of large inhomogeneous sheets of graphene in a tight binding model with Hubbard type interactions, where the exact functional was parametrised using Exact Diagonalisation data. This was demonstrated by using Exact Diagonalisation on the homogeneous 2D Hubbard model for small clusters of graphene flakes to construct an approximation to the exchange correlation potential. This representation of the functional was then used for much larger sizes of graphene systems including inhomogeneities and the results compared to predictions of the ground state by Lieb's theorem for Hubbard models on bipartite graphs.

As previously mentioned, there are many functionals which have impressive records across broad material classes when predicting certain properties, while completely failing for others. For example, many functionals tend to struggle with describing bond disassociation, i.e., when electrons localise on ions when they are upon stretching, and this is closely related to the misprediction of lattice constants in solids even when the electronic structure is qualitatively correct. Moreover, for strongly correlated materials systems the current generation of XC potentials often make qualitatively bad predictions for most electronic and structural ground state properties. In recent years exact many body corrections to DFT based on embedding schemes have demonstrated considerable promise. Marrying real space DFT with lattice Hamiltonians, in approximations such as the GW approximation or dynamical mean field theory, DMFT, is the current state of the art approach to modelling strongly correlated materials systems. In recent years, quantum many body approaches, such as DMFT, and quasiparticle self-consistent GW approaches have alleviated some of these issues in some strongly correlated systems, however they tackle the issue via exact embedding approaches. As the XC functional is not modified or updated, instead exact quantum simulations for restricted regions of the Hilbert space must be performed multiple times. Such exact quantum simulations require extensive classical computational power for each new material.

In addition, in recent years machine learning methods have been trained against exact results, showing utility for parametrising the XC functional for molecules and for the Hubbard model. Sequences of tests in 1d that explore the effect of impurities in these systems have been performed. It is now clear that improving methods for XC functional design is a key factor in unlocking the full range of capabilities afforded by Kohn-Sham DFT. Improvements to DFT computations have also been proposed using a technical approach where a local approximation of the XC functional is calculated based on the Zumbach Maschke basis, by performing density matrix measurements on a quantum computer. In this case, density matrix measurements means computing the one- and two-body reduced density matrices on a quantum computer. The one-body reduced density matrix is,

$${ρ}_{ij} = 〈 {Ψ}_{G S} \vert {c}_{i}^{†} {c}_{j} \vert {Ψ}_{G S} 〉$$

where |Ψ_{GS} [symbol] is the ground state. While the two-body reduced density matrix is given by:

$${Γ}_{ijkl} = 〈 {Ψ}_{G S} \vert {c}_{i}^{†} {c}_{k}^{†} {c}_{l} {c}_{j} \vert {Ψ}_{G S} 〉 .$$

The proposed method states that once the ground state is known and these measurements are performed then the XC potential can be estimated. Thus, the formulation of DFT in the Zumbach Maschke basis outlines a technique to compute a local approximation of the XC functional based on density matrix measurements.

For the fault tolerant framework, an approach has been proposed that could obtain the exact XC functional by using machine learning, but this requires the use of quantum algorithms that are not suited for near term devices. The Levy-Lieb formalism of DFT has been applied to the one dimensional Hubbard model using quantum algorithms, such as VQE, to compute the ground state properties of the Hubbard dimer, while supplementing the technique with a quantum kernel that can learn the density functionals of observables by machine learning. This approach focuses on how to apply machine learning to low dimensional functions, for example the Hubbard dimer, and may not be scalable to allow application to larger physical, material, chemical, or biological systems.

It is generally accepted that the computational efficiency of exact quantum simulations may be vastly improved through the use of reliable and efficient quantum computers that lack the drawbacks of currently available, and near-term, NISQ computers. Noisy intermediate-scale quantum processors are currently limited by significant issues with gate fidelity, and decoherence, which limit them to low numbers of qubits. In turn, this limits possible application to those which can be performed by quantum algorithms that are accurate for shallow-depth quantum circuits. Additionally, near term quantum algorithms such as VQE, may not succeed in producing good approximations to the ground state of interacting systems, in particular as the system becomes more parametrised. As such, the direct application of VQE to interacting systems using NISQ processors has caveats and uncertainties when computing ground state properties for simulation of real-world.

### SUMMARY OF THE INVENTION

Aspects and examples of the invention are set out in the claims and aim to address at least a part of the above described technical problem, and related problems.

In particular, the invention set out herein is directed to an improved DFT algorithm by utilising current and near-term NISQ processors and intentional simplification of the systems to be simulated and careful selection of which parts of the DFT algorithm are performed by quantum computation rather than classical computation in order to maximise the benefits of such a hybrid quantum-classical DFT algorithm. In this way, the methods can be thought of, in part, as providing a bridge between a complex system to be simulated on the one hand and a limited capacity and robustness in the otherwise seemingly ideal environment for simulations of quantum systems.

As will be apparent from the foregoing, this application discloses several advantageous approaches to performing DFT on a hybrid quantum-classical processing system. These include:

A hybrid quantum classical algorithm that uses low-depth and poor-quality quantum simulations to improve the quality of solutions over classical approximations for classical Density Functional Theory simulations.

A systematic method to design local quantum exchange correlation functionals for lattice models.

Quantum algorithms for the exact extraction of external potential to density mappings and the respective inversion.

Density constrained parametrised quantum circuits for inhomogeneous interacting fermionic systems.

A quantum kernel method for the extrapolation to the thermodynamic limit of the local density exchange correlation energy functional.

A hybrid quantum classical algorithm for the multivariate DFT XC functional through global perturbations via the external potential.

Disclosed herein is a method of performing improved Kohn-Sham Density Functional Theory, DFT, calculations, for simulation of a material, chemical, or biological system, comprising the steps of: (i) constructing a model Hamiltonian of the system to be simulated; (ii) inputting an electron density of the system to be simulated; (iii) constructing a Kohn-Sham potential by:
(1) providing a second Hamiltonian based on the model Hamiltonian;
(2) calculating a ground state energy of the second Hamiltonian, as a function of electron density, using a quantum algorithm executed by a noisy intermediate-scale quantum, NISQ, processor;
(3) computing a quantum-obtained exchange correlation energy functional, as a function of electron density, from the NISQ processor-obtained ground state energy of step (2);
(4) obtaining a quantum-obtained exchange correlation potential functional by differentiation of the quantum-obtained exchange correlation energy functional from step (3); (5) using the quantum-obtained exchange correlation potential functional from (4) to construct the Kohn-Sham potential; and
   (iv) executing a quantum Kohn-Sham DFT algorithm iteration using the electron density from (ii) and the Kohn-Sham potential from (iii) to compute an updated electron density and an updated total energy of the system to be simulated.

Advantageously, this method combines the merits of both DFT and quantum algorithms, by using results from a quantum algorithm to provide a qualitatively improved exchange correlation potential functional (XC functional) that can nudge the DFT algorithm towards more precise solutions. The improvement in exchange correlation functional arises from step (2) where the ground state computation is performed by quantum algorithms on an NISQ. Advantageously, this method relies only on measurements of the ground state energy to calculate an improved XC functional for use in step (iv) and does not explicitly require updates to the form of the XC functional iteratively based on quantum-computed density matrix measurements. Furthermore, as the computation of the ground state energy is the only step that must be carried out by an NISQ processor, the use of quantum resources can be minimized while still producing an improvement in the efficiency and accuracy of DFT simulations for a wide variety systems that are traditionally difficult to simulate using DFT. Additionally, low quality quantum algorithms can be successfully used in this way meaning that this method can be executed employing NISQ processors with limited numbers of qubits rather than needing to wait for further innovation in quantum computation hardware. Once the ground state energy has been computed, the quantum-obtained XC potential can be used to construct a Kohn-Sham potential and provided to a DFT calculation on a classical computer for any target molecular system and this can be used to estimate ground state properties such as energies, forces, and densities. These ground state properties may then ultimately be used to inform higher level quantum mechanics (QM) and molecular mechanics (MM) simulations. Furthermore, this method is flexible and capable of scaling with the size of available quantum processor, whereas the classical approaches to approximating the exchange correlation can only be applied for small system sizes. Specifically, it is possible to design an approximation to the XC potential using a system which is not the same size—importantly the system may be orders of magnitude smaller than the target system. For example, the exchange correlation functional from a 1×50 simulation may be used within the DFT simulation of a 1×1000 system and still have the ability to nudge the DFT variational algorithm towards more precise solutions. This represents a significantly more efficient method for DFT simulation of large systems. Although inherent finite-size errors may arise from the use of a 1×50 functional in this context, it may still be used and prove competitive against state-of-the-art classical methods of approximating the exchange correlation functional. In this sense, we incorporate into the approach both that within a NISQ environment only low depth and small qubit cases are possible.

Advantageously, this method can be applied to periodic varying potentials—which covers all solids in their metallic, insulating, semiconducting and superconducting phases—including defect driven or doped systems. Furthermore, strongly correlated electron systems, which are difficult to deal with by classical DFT methods, can also be simulated with this improved method of DFT.

Advantageously, DFT in the Kohn Sham formulation implemented as a self-consistent variational algorithm is amenable to dealing with a certain level of error in the approximation of the XC potential. This means that even if the XC potential is quantitatively erroneous for certain density values, if it is qualitatively accurate near the ground state then it will evaluate the ground state energy and density accurately there. If at a single iteration the evaluation of the XC potential is incorrect, the next iteration may this corrects for itself. The relatively high tolerance of Kohn-Sham DFT to quantitative errors in XC potential means that this method is robust and more tolerant of potentially noisy estimates of the ground state calculated using NISQ processors.

Optionally, providing the model Hamiltonian in step (i) includes either: providing an inhomogeneous Hamiltonian; or providing a homogeneous Hamiltonian, wherein the homogeneous Hamiltonian is the inhomogeneous Hamiltonian with an external potential removed.

Advantageously, this method of DFT can be applied to simulate systems that are homogeneous and inhomogeneous. This allows for complex systems with non-zero external potentials to be modelled.

Optionally, when the model Hamiltonian provided in step (i) is inhomogeneous then the second Hamiltonian provided in step (1) is homogeneous, further wherein the model Hamiltonian has an external potential and the second Hamiltonian is based on the model Hamiltonian with the external potential removed.

When the model Hamiltonian of the system to be simulated is inhomogeneous (for example in strongly correlated electron systems), the qualitative features of the XC functional can be approximated well by calculation of an XC functional from the ground state of a homogeneous version of the model Hamiltonian that has been calculated using a quantum algorithm as in step (2). By providing a homogeneous Hamiltonian, related to the inhomogeneous Hamiltonian by removal of an external potential term, in step (1) it is possible to reduce the complexity of the quantum ground state calculation while still being able to calculate an improved XC functional that is capable of steering DFT simulations of the inhomogeneous system. Because the method is robust against quantitative errors in XC functional and the improvements arise from improved qualitative XC functionals improvements to DFT can be achieved even on current NISQ processors which frequently do not have the gate fidelity to achieve quantitatively accurate simulation of even homogeneous systems directly.

Optionally, calculating the ground state of the second Hamiltonian on an NISQ processor further comprises using a fermion-to-qubit encoding to encode qubits of the NISQ processor.

The fermion-to-qubit encoding can be chosen from a number of possible encodings—e.g. Jordan-Wigner, compact, and Bravi-Kitaev—to streamline encoding of the NISQ processor based on the form of the second Hamiltonian and the NISQ processor hardware. The encoding defines how fermion operators are mapped into qubit operators which manifest as gates in the quantum circuit.

Optionally, the fermion-to-qubit encoding is a Jordan Wigner encoding that maps fermionic creation and annihilation operators of the second Hamiltonian to Pauli strings comprising Pauli X, Y and Z operators.

Jordan-Wigner encoding is a well-known fermion-to-qubit mapping which allows for easy conversion of known model Hamiltonians, e.g., the Hubbard model, into Pauli operators that can be easily converted into a quantum circuit. This encoding is compatible with current and near term NISQ processors because it is well understood how to convert Pauli operators into quantum gates, such as CNOT and RZ gates, which are commonly native to NISQ hardware. This is convenient because it can reduce the amount computational processing required to represent Hamiltonians as appropriate quantum circuits. Conventional Jordan Wigner mapping associates each fermionic mode with a qubit.

Optionally, calculating the ground state of the second Hamiltonian on an NISQ processor further comprises enacting qubit operations generated by unitary qubit operators on the qubits of the quantum information processor.

In order to compute ground state energies for these models XC potentials are created that can be used in the DFT algorithm. Quantum algorithm are used to determine the order and on what qubits the gates which appear in the circuit are executed. A quantum calculation of the ground state comprises the complete execution of a quantum circuit (a way of representing the qubit operations and the qubits on which they act during the algorithm) after which an observable is measured, in this case the ground state energy. The encoding defines how fermion operators are mapped into qubit operators which manifest as gates in the quantum circuit. A realistic practical assumption may be that CNOT and RZ gates are native on the hardware device. The second Hamiltonian, in terms of Pauli strings, may therefore be decomposed into a sequence of CNOT and RZ gates.

Optionally, the model Hamiltonian is the Hubbard model with an external potential term, and the second Hamiltonian is the Hubbard model.

Using DFT to study model Hamiltonians such as the Hubbard model, is useful for a number of reasons, chief among them being that model Hamiltonians already have exact solutions (for small system sizes or certain dimensions), so it is straightforward to assess the quality of a new approximation. The nomenclature of using inhomogeneous for the Hubbard model and an external potential can be related to the similar use of describing materials by an inhomogeneous electron gas.

Optionally, execution of the quantum algorithm by a noisy intermediate-scale quantum, NISQ, processor further comprises executing a Variational Quantum Eigensolver, VQE, algorithm using the NISQ processor.

VQE is a hybrid quantum-classical algorithm for approximating ground state energies on quantum computers. While it is possible to use other quantum algorithms, including for example the Dissipative Quantum Eigensolver (a method for provably approaching a known, e.g. ground, quantum state of an encoded Hamiltonian), VQE has particularly good performance for standard homogeneous Hamiltonians like the Hubbard model. The QC gates are determined by a combination of the algorithm selected for approximating the ground state energy, the choice of fermion-to-qubit encoding, and the particular material or molecule the method is being performed on. As it is well known how to prepare Hamiltonians for simulation using VQE this is a convenient choice.

For VQE we use the standard options as mentioned above. We emphasise however that, unlike when solving problems directly using VQE, we do not necessarily need very accurate approximations for the novel methods disclosed herein, so we can choose lower-depth VQE leveraging the synergy between the classical and quantum systems as explained above, and still obtain good results, where VQE alone at that circuit depth performs poorly or is even provably incapable of finding the true ground state and ground state energy.

To measure the ground state energy using a quantum algorithm we choose VQE, but in general are not restricted to doing so. To deal with continuous systems the VQE circuit can be changed to reflect the internal symmetries of the considered materials system. So, for example, modelling graphene would require a Hubbard model with next-nearest neighbour hopping on a hexagonal lattice. In practice these are all accounted for in the current generations of VQE algorithms. Advantageously, VQE evaluation of an XC functional qualitatively captures some of the main features of the XC potential, even where it is not quantitatively accurate. An important example of these qualitative features is whether or not the XC functional has a discontinuity. More generally, any approximation to the XC potential that incorporates the effects of exchange and correlation of fermions may succeed in nudging the DFT algorithm to more accurate solutions in strongly interacting parameter regimes, with respect to the functionals that do not model exchange or correlation. It is also possible to incorporate constraints into the XC potential that are already known. For example, if the considered system has a fundamental gap or is fully occupied, it is possible to include extra data to the XC potential functions that take advantage of symmetries and known properties of the system. In other words, it is possible to introduce error mitigation techniques when constructing the XC functional obtained from VQE simulations. These techniques effectively reduce the sensitivity of the quantum algorithm to noise, as well reasoned and physically motivated post processing on the quantum data can be performed.

Advantageously, it is possible to use quantum simulated ground state data from low depth VQE circuits and for small lattice sizes and then encode that information into the exchange correlation functional that be used for much larger system sizes, i.e., it is independent of system size. In doing so, it is also possible to systematically improve the approximation by extrapolating away finite sizes effects, incorporate constraints that must be respected and improve with deeper circuits.

Optionally, the VQE algorithm is implemented as a VQE circuit which is lower depth than VQE circuits required to find the ground state of the model Hamiltonian directly.

Advantageously, low quality and low depth data (both from simulations and real quantum computer runs) can be used to improve DFT simulations, even though the quantum approximations themselves are low quality and from low-depth computations on noisy, small-scale quantum hardware. This method advantageously allows DFT simulation of systems with model Hamiltonians that are too complex for direct solution with VQE on current and near-term NISQ processors due to decoherence limitations. It is surprising that a noisy NISQ processor computed ground state of the second Hamiltonian can still produce a DFT output that falls within 1% of in particular parameter regimes of the Hubbard model. This is in contrast to the classical mean field results, which fall outside of this 1% interval. Additionally, the approach does reasonably well at capturing qualitative features of the XC potential as compared to exact classical methods—in particular, the discontinuity of the XC potential is well captured by VQE methods. The XC potential in the Hartree Fock approximation by contrast, perhaps the most famous single particle method after DFT, is zero everywhere by definition, so it is impossible to infer how Mott physics appears in the functional with Hartree Fock. Advantageously, for strongly correlated systems

$$( \frac{U}{t} > 1 )$$

the estimation of the density is also well described.

Optionally, the VQE algorithm uses a Hamiltonian Variational Ansatz, HVA.

There are different possible VQE ansatzes, the inventors have chosen the popular Hamiltonian Variational Ansatz (HVA) for VQE simulations. For HVA, the initial state can be advantageously prepared from the ∪=0 ground state of the Hubbard model, which comprises a layer of depth nₓn_{y}−1 of Givens rotations acting on adjacent modes. The decomposition of the initial state into signals depends on the native gates available on the hardware device. nₓ; n_{y} are the system dimensions, e.g here we consider 2d. The HVA may also be updated with single qubit rotation gates when an inhomogeneous model is under consideration, as opposed to just taking the free fermion terms, for example if the second Hamiltonian in step (2) is inhomogeneous.

Optionally, obtaining a quantum-obtained exchange correlation potential functional in step (4) further comprises using a quantum kernel method for continuous representation of the exchange correlation energy functional as a function of electron density.

This allows for continuous representations of the exchange correlation potential, which can be more easily applied to large systems, whilst reducing finite size effects. Optionally, the quantum kernel method comprises interpolative and/or extrapolative procedures for obtaining the exchange correlation energy functional as a function of electron density.

Interpolative and/or extrapolative procedures are well known and already optimised procedures that allow for construction of continuous representations. In this case, continuous representations of the exchange correlation potential, which can then be more easily applied to large or complex systems, whilst reducing finite size effects. Using interpolative and/or extrapolative procedures also allows for a qualitatively improved XC functional while being able to modify the number of required VQE/quantum algorithm calls on the NISQ processor. The maximum number of calls is related to the size of the system to be simulated and the number of system sizes being considered.

Optionally, the quantum kernel method further comprises using a Local Density Approximation, LDA, to construct a continuous representation of the exchange correlation energy functional.

In the classical method of DFT for solids, the Local Density Approximation (LDA) uses the Quantum Monte Carlo simulations of the homogeneous electron gas to approximate the XC functional, and then uses DFT to (in theory) solve the interacting inhomogeneous (due to the presence of an external potential) electron gas model. The application of LDA to the XC energy functional in step (4) expresses the XC energy functional in a compact representation so that the XC potential functional can be easily computed by differentiation, i.e.

$${{V}_{X C}^{Q L D A} ( {n}_{i} , U ) = \frac{\partial {E}_{X C}^{Q L D A} ( n , U )}{\partial n} \vert}_{n = {n}_{i}} .$$

Thus, for a system of size L, n₁ ranges from n₁ to n_{L} and the maximum number of VQE calls to approximate the XC energy functional is given by 2L. By using interpolative and extrapolative techniques it is possible to modify the number of these calls, whose upper bound will be 2ΠᵢLᵢ, where Lᵢ is the set containing the number of system sizes considered, e.g. L={4, 5, 6} would require at most 240 quantum computations to be performed.

Optionally, the material system to be simulated is a strongly correlated electron system.

This method is particularly well suited to improvements in simulating strongly correlated electron systems because it is possible to use a simple model incorporating Mott physics as the second Hamiltonian in order to obtain a much-improved qualitative XC potential functional that includes essential features, such as discontinuities, needed for simulating such systems. Additionally, simulation of complex, real-world systems generally requires the ability to incorporate the properties of strongly correlated electrons which has been a previous failure of DFT due to inability to produce quality XC potential functional for such systems.

Optionally, the Kohn-Sham DFT algorithm is a Kohn-Sham Lattice Density Functional Theory, LDFT, algorithm.

An advantage of considering lattice models over the electronic Hamiltonian is that, for electronic Hamiltonians, a physically reasoned basis must be chosen for the system at hand, and this introduces additional, complicated, technical considerations. Additionally, model Hamiltonians already have exact solutions (for small system sizes or certain dimensions), so it is straightforward to assess the quality of a new method of exchange correlation functional approximation. However, the algorithmic procedures for lattice models and electronic Hamiltonians are equivalent and either may be used with the method described herein.

Optionally, the NISQ processor requires fewer qubits to calculate the ground state of the second Hamiltonian than the number of qubits required to calculate the ground state of the model Hamiltonian of the system to be simulated directly.

By reformulating the model Hamiltonian as a second Hamiltonian for which the ground state is calculated, it is possible to reduce the quantum resources required for this method. This is advantageous because it means that a significantly improved method of DFT can be implemented using current and near term NISQ processors rather than needing to wait for significant advances in hardware for quantum computation.

Optionally, a quantum Kohn-Sham DFT algorithm iteration in step (iv) comprises the steps of
(a) taking the electron density from (ii) and the Kohn-Sham potential from (iii) as an input to construct Kohn-Sham equations;
(b) solving the Kohn-Sham equations;
(c) providing the updated electron density, and the updated total energy of the system to be simulated, based on solutions of the Kohn-Sham equations in step (b); and
(d) checking if a convergence condition is met, further wherein the condition for convergence is evaluated based on the electron density input in step (ii) and the updated electron density.

Regarding the ground state problem and DFT, the ground-state density minimises the total energy functional which enforces the functional derivative to be zero and results in a variational equation for wavefunctions that can construct the ground state density. This equation takes the form of a single-particle Schrodinger equation which can be treated as a standard eigenvalue problem. All of many-body physics manifest in the quantum-computed exchange correlation potential contribution of an otherwise fully determined effective single particle potential. These equations are solved for self consistently, as the XC potential depends on the density, and the density depends on the unknown wavefunctions. At self-consistency, these wavefunctions no longer change and are used to construct the ground state density and energy.

Optionally, evaluating the convergence condition in step (d) comprises calculating:
(α) a density difference metric from the electron density input in step (ii) and the updated electron density; and/or
(β) an energy difference metric from a total energy of the system to be simulated, based on the electron density from step (ii), and the updated total energy of the system to be simulated,
and comparing the density difference metric from (a) and/or the density difference metric from (D) with a threshold condition for each difference metric.

A possible density difference metric is the L2 norm of density vectors, given by

$$Δρ = \frac{1}{L} {\sum }_{j}^{L} \vert {ρ}_{j , current} - {ρ}_{j , previous} \vert .$$

A reasonable energy difference metric may be ΔE=|E_{updated}−E_{step (ii)}|, and the threshold condition may then take the form of asking if ΔE<X, then convergence is considered to be met and the iterative stops. X is typically less than 10⁻⁸.

Optionally, further quantum Kohn-Sham DFT algorithm iterations, are performed by providing the updated electron density from (c) as the input in step (ii) if the convergence condition in (d) is not met such that steps (ii) to (iv) continue until the convergence condition for the electron density, and/or the convergence condition for the total energy of the system to be simulated, is met in step (d).

The manner in which we have implemented the procedure allows for a clean separation of the quantum and the classical processing, so that the method is not dependent in any way on fast communication between the classical and quantum parts. However, we also highlight that there are alternative implementations of our method that would require on-the-fly calls to the quantum computer. In the workflow where the quantum computer, or NISQ processor is called at each cycle of the classical DFT algorithm, this would necessarily need to be automated. In this case at each iterative step of the DFT algorithm the XC potential must be queried for a given density and this evaluation is performed on-the-fly.

Optionally, a further Kohn-Sham DFT iteration is performed in a classical manner, the classical Kohn-Sham DFT iteration comprising the steps (ii) to (iv), wherein step (ii) receives the updated electron density from (c) as an input and step (iii) replaces steps (1) to (5) by providing a classically obtained exchange correlation potential functional as an input to construct the Kohn-Sham potential.

An advantage of this approach is that it combines classical and quantum iterations to reduce the number of calls to NISQ but can still yield an unexpectedly improved accuracy.

Optionally, further classical Kohn-Sham DFT iterations are performed until the convergence condition for the electron density, and/or the convergence condition for the total energy of the system to be simulated, is met in step (d).

This improved method of DFT can provide a significant advantage over methods that employ traditional XC functional approximations, even when only one quantum DFT is performed, and the remaining iterations performed until convergence is deems to be met. This allows for the number quantum computing resources to be minimised. Thus, this method of improved DFT can be realised even with in the current limitations in quantum computation hardware.

Optionally, further quantum or classical Kohn-Sham DFT algorithm iterations are performed, including at least one further quantum Kohn-Sham DFT iteration, until the convergence condition for the electron density, and/or the convergence condition for the total energy of the system to be simulated, is met in step (d).

Further quantum DFT iterations may be executed, to allow for simulation of more complicated material, chemical, and biological systems where it may be necessary to nudge the XC potential functional more than once to improve the accuracy of the simulation. In this way, the method can be optimised for accuracy and computational efficiency, allowing for simulations that are as fast as possible without sacrificing accuracy.

Optionally, further quantum Kohn-Sham DFT iterations are performed at periodic intervals with classical Kohn-Sham DFT algorithm iterations in between, until the convergence condition for the electron density, and/or the convergence condition for the total energy of the system to be simulated, is met in step (d).

We note that in all of these formulations it is also possible, and may be advantageous, to “toggle” the calls to the quantum computer, e.g. maybe the QC is only called every 10 DFT iterations, rather than every single iteration, to reduce the total number of calls to the quantum computer.

Optionally, the updated electron density, and the updated total energy of the system to be simulated, from step (c) are used to construct ground state properties of the model Hamiltonian describing the material system which is to be simulated.

Knowledge of the ground state properties of the model Hamiltonian allows for calculation of many different properties of a material, chemical, or biological systems. Some examples of such properties include energies, forces, and densities which influence the behaviours of these systems. In addition to materials modelling, qDFT could also be applied to quantum chemical systems. By using qDFT as a subroutine to predict ground state properties it could also be used for Molecular Dynamics (MD) and structure searching applications, including simulation of biological systems such as covalent inhibitors in Myotonic Dystrophy applications.

Optionally, the method may comprise, prior to steps (i) to (iv), the step of performing at least one classical Kohn-Sham DFT algorithm iteration using a classical exchange correlation potential functional.

This method may also be advantageously combined with existing methods for DFT.

Also disclosed herein is a hybrid quantum-classical apparatus for simulation of a material, chemical, or biological system, by way of enacting the above described method.

The method is not a priori tied the hardware details. However, as with all quantum computation methods for materials or chemistry systems, for any concrete material or molecule it is necessary (or at least highly advantageous) to match the interaction structure of the material or model to the interaction structure of the qubits in the quantum computer.

Optionally, the NISQ processor may be a Superconducting qubit based platform. These platforms have the most impressive results for large system sizes, but this is because other platforms are restricted to small qubit numbers at the moment. As VQE is a quantum-classical hybrid method, it communicates between the quantum computer to the classical computer to update the weights of the parametrised quantum circuit, as determined by the choice of encoding and ansatz.

The parameters one needs to know about a quantum device are its connectivity, native gates, and number of available qubits for computation. The resulting VQE circuit encapsulates the encoding and initial state that is run on the NISQ processor. The parameters needed to set up NISQ hardware for a specific VQE process is the representation of the fermionic Hamiltonian decomposed into native gates on that processor. As VQE is a hybrid-classical algorithm, this VQE circuit is parametrised by variational parameters which are updated using a classical optimised algorithm run on a classical computer between each query to the quantum computer. Here the classical optimiser which is used is crucial in ensuring the success of a NISQ computation, but standard off-the-shelf classical optimizers can be used for this purpose. Furthermore, it is possible to also tailor the choice of decomposition into QC gates to the specific quantum hardware capabilities, for example qubit layout, connectivity.

Optionally, the hybrid quantum-classical apparatus may comprise:
a noisy intermediate-scale quantum, NISQ, processor; and
at least one classical processor,
wherein the NISQ processor is used to calculate the ground state energy of the second Hamiltonian in step (2). Optionally, the at least one classical processor is used to perform the quantum Kohn-Sham DFT iterations in step (iv).

Optionally, the at least one classical processor is used to perform the classical Kohn-Sham DFT iteration(s).

Optionally, the at least one classical processor is used to perform all of the steps of the disclosed method except for step (2).

Near term quantum methods can greatly benefit from an efficient quantum-classical algorithmic protocol, as these devices are restricted in the number of qubits available and circuit depth achievable before decoherence of the qubits occurs, rendering the simulation overcome by noise. Here we follow an approach where the quantum device produces classical data which informs a classical algorithm that is run on a classical computer, so the computational load is optimally split between classical and quantum resources. In particular, we use the quantum computer to produce an approximation to the exact exchange correlation energy for molecular systems, which can be used to create classical representations of the exchange correlation potential required within a DFT algorithm.

Other steps may also be performed on the NISQ processor, as appropriate to the capability of the hardware. More than one NISQ processor may be utilised, and/or more than one classical processor may be used.

Optionally, the at least one classical processor performs steps comprising at least one of:
a fermion-to-qubit mapping of the second Hamiltonian into a parameterised quantum circuit;
updating VQE parameters based on the variational principle;
obtaining a continuous representation of the exchange correlation energy functional or the exchange correlation potential functional;
storing an exchange correlation potential functional locally or transmitting it to a remote location; or
storing an exchange correlation energy functional locally or transmitting it to a remote location.

Storing the exchange correlation potential functional locally or transmitting it to a remote location allows for this method to be optionally implemented with a clean separation between the quantum executed processes and the classically executed processes. For example, software may be used to compute the ground state energy value at a given density, for a Hamiltonian which may be passed to an automatic internal library that is fully automated to return the ground state energy, or the exchange correlation potential functional, or the exchange correlation energy and density for a given external potential.

Optionally, at least one classical processor is part of a control apparatus for the NISQ processor.

Also disclosed herein is a non-transient computer readable medium comprising instructions which cause a computer to enact the above-described method steps.

Initialisation of the quantum circuits may be automated via software, or it can be performed semi-manually. In the workflow where the XC functional is computed in advance and stored for later lookup, it could be done semi-manually, though automating it would be convenient.

The quantum computer is used to construct an approximation to the exact exchange correlation potential. This does not have to be computed in real-time during the DFT algorithm. It could also be precomputed and then stored in a lookup table or database, that can then be used by a classical DFT algorithm. DFT is a variational algorithm and thus is iterative, so at each step of the DFT cycle a query to this database is performed or the quantum computer is called directly.

The main idea that we propose here is to combine the merits of both classical DFT with near term quantum algorithms such as VQE. The essence of the approach is to bootstrap DFT with low-depth and poor-precision VQE simulations to construct quantum enhanced XC functionals. By doing so we nudge the classical DFT method towards better solutions than those obtained with classical functional approximations. The merits of this approach compared to state-of-the-art classical functionals are shown by applying it to the Hubbard model in a number of parameter regimes and dimensionalities.

### BRIEF DESCRIPTION OF THE FIGURES

FIG. 1A is a flow chart illustrating steps in a hybrid method for generating an XC functional as applied to the specific example of the Hubbard dimer, including equations and code for each step;

FIG. 1B shows images corresponding to each of the steps in FIG. 1A;

FIG. 2A shows a flow chart for a DFT loop as part of the methods disclosed herein, as applied to the specific example of the Hubbard dimer, including equations and code for each step;

FIG. 2B shows images corresponding to the steps in FIG. 2A;

FIG. 3 shows an overview of the hybrid quantum-classical DFT process;

FIG. 4 shows representative data for the energy per site computed using a homogeneous Hamiltonian in the DFT loop;

FIG. 5 is the exchange correlation energy calculated from FIG. 4;

FIG. 6 shows representative data for the ground state energy, highlighting the convergence within DFT;

FIG. 7 shows representative data of the ground state density for an example of a 1D Hubbard model as a function of U using various models;

FIG. 8 illustrates a trade-off between complexity and computational complexity often encountered in DFT;

FIG. 9 shows a quantum circuit for performing VQE;

FIG. 10 shows a plot of a universal functional within the Kohn-Sham theorem for U=5;

FIG. 11 shows a plot of potential-density invertibility within the Kohn-Sham theorem for U=5;

FIG. 12 shows the homogeneous energy modelled by various methods;

FIG. 13 shows the correlation energy of the same system as FIG. 12 modelled by various methods;

FIG. 14 shows the kinetic energy of the same system as FIG. 12 modelled by various methods;

FIG. 15 shows the Hartree-Fock energy of the same system as FIG. 12 modelled by various methods;

FIG. 16 shows the XC potential of the same system as FIG. 12 modelled by various methods;

FIG. 17 shows a comparison between different models for calculating an electron density of a small 1D chain;

FIG. 18 shows the difference between the ground state density for the exact result and various methods shown in FIG. 17 without constraints;

FIG. 19 shows a comparison of three methods after 5 Kohn-Sham iterations;

FIGS. 20 and 21 show various parameters as calculated after 5 Kohn-Sham iterations for BALDA and VQE respectively;

FIG. 22 shows the difference between the ground state density for the exact result and various methods;

FIG. 23 is a schematic of a quantum-classical hybrid algorithm to accelerate and improve over standard classical methods for the simulation of covalent inhibitors in Myotonic Dystrophy applications;

FIG. 24 is a schematic showing the interrelationships between various subroutines in the overall process;

FIG. 25 is a schematic illustrating the algorithmic workflow of quantum computation within a quantum/materials modelling calculation;

FIG. 26 shows the XC energy (left) and potential (right) for the 1×12 Fermi-Hubbard model at U/t=4 using different methods. The solid lines are produced using a cubic spline. The VQE results are obtained using a classical emulator. Note that the continuous LDA approximations perform badly in this system.

FIG. 27 shows the effect of U and system size on the XC potential for 1D Fermi Hubbard models.

FIG. 28 shows the log₁₀ Frobenius norm error between the VQE XC potential functionals and the exact functional for 1D Fermi-Hubbard models at different values of interaction strength U/t and system size nₓ.

FIG. 29 shows the effect of U and system size on the XC potential for 2D Fermi Hubbard models.

FIG. 30 shows the log₁₀ Frobenius norm error between the VQE XC potential functionals and the exact functional for 2d Fermi-Hubbard models at different values of interaction strength U/t and system size [nₓ, n_{y}].

FIG. 31 show the 1×12 Fermi Hubbard model for U=4 and at quarter-filling with an external quadratic potential. FIG. 31A illustrates the convergence of the total energy per lattice site within the DFT loop. FIG. 31B displays the density as predicted using different DFT functionals and the corresponding exact methods (ED/VQE).

FIGS. 32A-C show the 1×12 Fermi Hubbard model for U=4 and at quarter-filling for different external potentials (in each of FIGS. 32A-C, where FIG. 32A has no external potential, FIG. 32B has a quadratic potential, and FIG. 32C has an impurity potential). The first row in each of FIGS. 32A-C compares the DFT densities compared to the exact predictions. The second row in each of FIGS. 32A-C monitors the convergence of the difference in total energy against the exact solution over the duration of the DFT computation. The third row in each of FIGS. 32A-C monitors the convergence of the difference in total density against the exact solution over the duration of the DFT computation.

FIG. 33 shows the 1×12 Fermi Hubbard model at quarter-filling with a quadratic external potential and its respective errors in energy (left panel) and density (right panel) for different interaction strengths. All simulations use DFT apart from the dashed lines.

FIG. 34 shows the performance of the finite size scaling of XC functionals in the convergence of the total energy error (top panel) and density error (bottom panel) with respect to the exact solution for the quadratic external potential. The left column shows the results produced with 1×6 XC potential. The right column shows the results produced with 1×12 XC potential.

FIGS. 35A and 35B show the 1×200 Fermi Hubbard model for U=10 and at quarter filling within a confining quadratic potential using a QEDFT functional generated on a 1×12 homogeneous Fermi-Hubbard system. FIG. 35A illustrates the convergence of the total energy per lattice site within the DFT loop. FIG. 35B, the density as predicted using different DFT functionals.

FIG. 36 shows the 1×200 Fermi Hubbard model at quarter-filling with a quadratic external potential and its respective errors in energy (left panel) and density (right panel) for different interaction strengths. All simulations use DFT and the BALDA DFT functional is used as the exact benchmark.

FIG. 37 shows the DFT energy convergence 3×3 Fermi Hubbard model for U/t=10 and at quarter-filling for no external potential and an impurity potential at the (1,2) site. Dashed lines represent non DFT calculations, which are either the exact solution or pure VQE emulations.

FIG. 38 shows the per site density percentage error for the 3×3 Fermi Hubbard model with impurity at site (1,2) for U/t=8. Pure VQE methods are highlighted in the top panel, while the other methods use DFT.

FIG. 39 shows the 3×3 Fermi Hubbard model at quarter-filling with an impurity potential at the (1, 2) site and its respective errors in energy (left panel) and density (right panel) for different interaction strengths. All simulations use DFT apart from the dashed lines.

FIG. 40 shows the performance of the finite size scaling of XC functionals in the convergence of the total energy error with respect to the exact solution for an impurity at site (1,2). The left panel shows the results produced with 2×2 XC potential. The right shows the results produced with 2×4 XC potential.

FIG. 41 shows the density per site of the 20×20 Fermi Hubbard model at half-filling at U/t=4 based on DFT calculations using functionals generated on a 3×3 lattice. The external potential is an impurity potential that concentrates fermions to a square at the centre of the lattice.

FIG. 42 shows the effect of system size on the XC potential for 1D and 2D Fermi Hubbard models at U/t=4 run on quantum hardware and classical simulations.

FIG. 43 shows the 1×8 Fermi Hubbard model for U=4 and at quarter-filling for different external potentials (FIG. 43A shows no external potential, FIG. 43B shows a quadratic external potential, and FIG. 43C shows an impurity potential) including results on hardware. The first (top) row in each of FIGS. 43A-C compares the DFT densities compared to the exact predictions. The second (middle) row in each of FIGS. 43A-C monitors the convergence of the difference in total energy against the exact solution over the duration of the DFT computation. The third (bottom) row in each of FIGS. 43A-C monitors the convergence of the difference in total density against the exact solution over the duration of the DFT computation.

FIG. 44 shows the 1×200 Fermi Hubbard model for U=4 and at quarter filling within a confining quadratic potential using a QEDFT functional generated on a 1×8 homogeneous Fermi-Hubbard system using hardware and emulation the density as predicted using different DFT functionals.

FIG. 45 shows the DFT energy convergence 2×4 Fermi Hubbard model for U/t=4 and at quarter-filling for no external potential and an impurity potential at the (1,1) site using the hardware data. Dashed lines represent non DFT calculations, which are either the exact solution or pure VQE emulations.

FIG. 46 shows the per site density percentage error (equation (65)) for the quarter-filled 2×4 Fermi Hubbard model with impurity at site (1,2) for U/t=4 including hardware data. Pure VQE methods are highlighted in the top panel, while the other methods use DFT.

### DETAILED DESCRIPTION

### A Quantum Density Functional Theory for the Hubbard Model

In this work, we approach the problem of XC functional design using quantum algorithms running on quantum computers.

Here we present a general hybrid-quantum algorithm in the context of materials simulation. It is applied to lattice models where it systematically uses VQE data to parametrise the functional form of the exchange correlation potential that is incorporated within a classical iterative Kohn-Sham scheme. By doing so, we show that in this hybrid quantum-classical DFT approach can be more accurate than the gold standard classical mean field approaches.

By way of example, we deal with materials and/or molecules which can be represented by fermionic lattice models such as the Fermi-Hubbard model. We present this approach in the context of the Inhomogeneous Hubbard Model for varying system sizes. With this technique it is possible to use quantum simulated data from low depth circuits and for small lattice sizes and then encode that information into the exchange correlation functional that be used for much larger system sizes, i.e., it is independent of system size. In doing so, it is also possible to systematically improve the approximation by extrapolating away finite sizes effects, incorporate constraints that must be respected and improve with deeper circuits. For materials systems believed to be appropriately modelled by lattice Hubbard Models this technique can efficiently solve much larger systems than with classically exact methods, in challenging regimes such as defect-driven phenomena and strongly correlated interactions.

Near-term hybrid-quantum algorithms that can compute the ground-state properties for fermionic systems have been produced. Our method uses quantum measurements from a state prepared on a quantum computer, that approximates the ground state of the system to establish a systematic scheme that computes exact exchange correlation functionals. To illustrate the technique, we use the Variational Quantum Eigensolver applied to the homogeneous and inhomogenous Hubbard model as a function of system size, lattice connectivity, electron doping, external potential and interaction strength. We show that low-depth VQE calculations of small system sizes are able to parametrise approximations of the exchange correlation functional, which can be used for systems of equivalent dimension but also much larger sizes. Furthermore, these functionals are used in the variational self-consistent scheme of Kohn Sham to produce the ground state properties of the interacting many-body system to a better approximation than classical mean field approaches in particular parameter regimes. Finally, we also illustrate how our approach can be extended to larger systems with varying connectivity structures that incorporate multiorbital electronic interactions.

Application to the Hubbard dimer

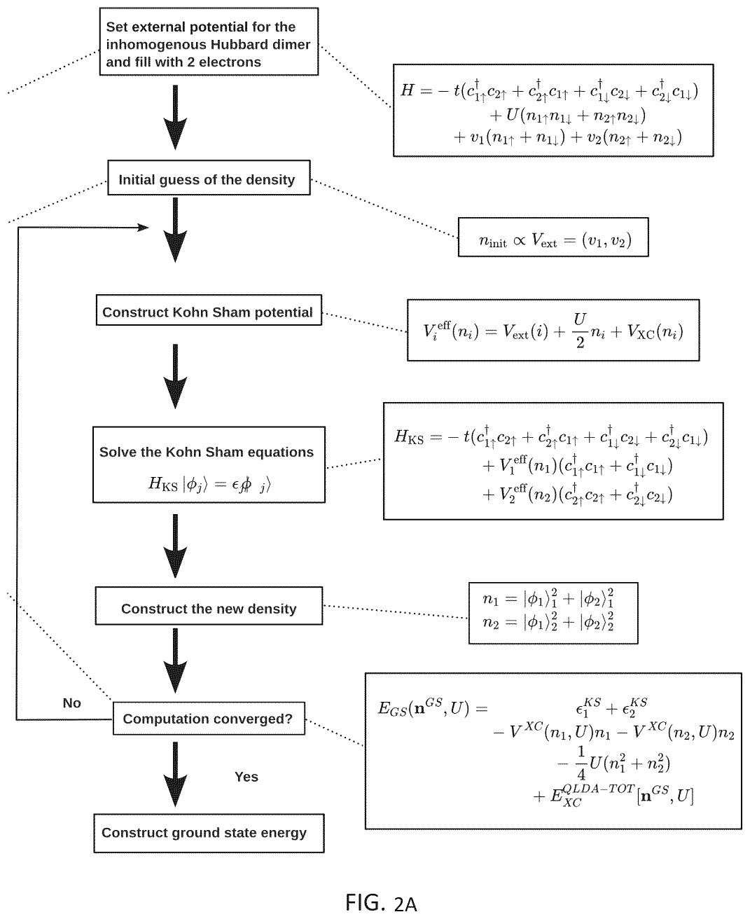

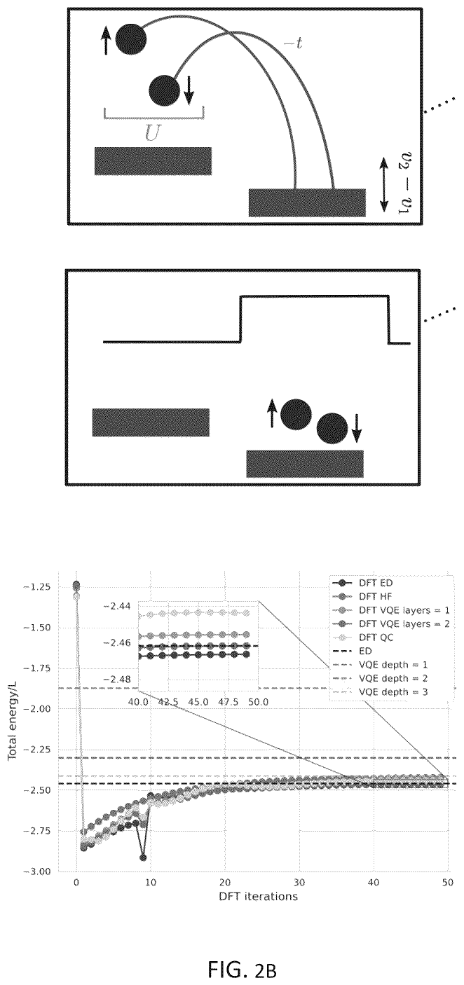

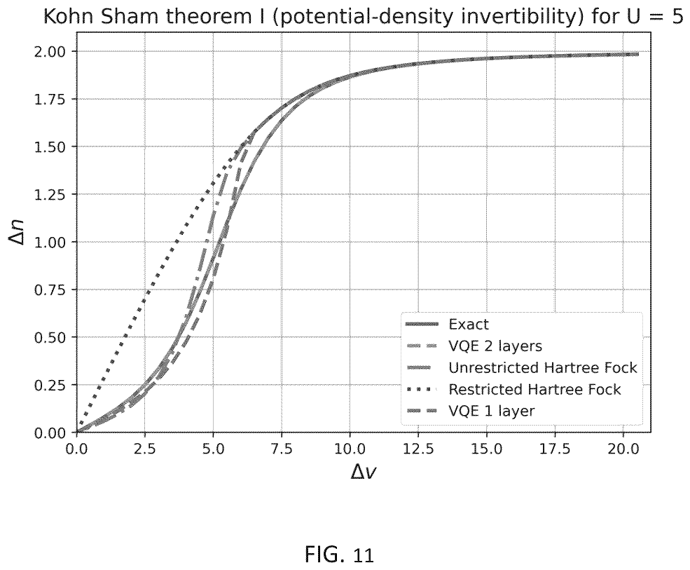

The Hubbard dimer is the simplest model to which this process can be applied. In FIG. 11 we show the process within a specifically designed flow chart for the Hubbard dimer of how to compute the XC potential, while in FIG. 2 we show how to incorporate that functional into a classical DFT loop for the dimer.

Taking the process step by step, we have that:
(a) The ground state energy E_{dimer} of the Hubbard dimer (e.g. the second Hamiltonian),

$${H}_{d i m e r} = - t {\sum }_{i σ}^{1} ( {c}_{i , σ}^{†} {c}_{i + 1 , σ} + {c}_{i + 1 , σ}^{†} {c}_{i , σ} ) + {\sum }_{i = 1}^{2} U {n}_{i , ↑} {n}_{i , ↓} \qquad [ 1 ]$$

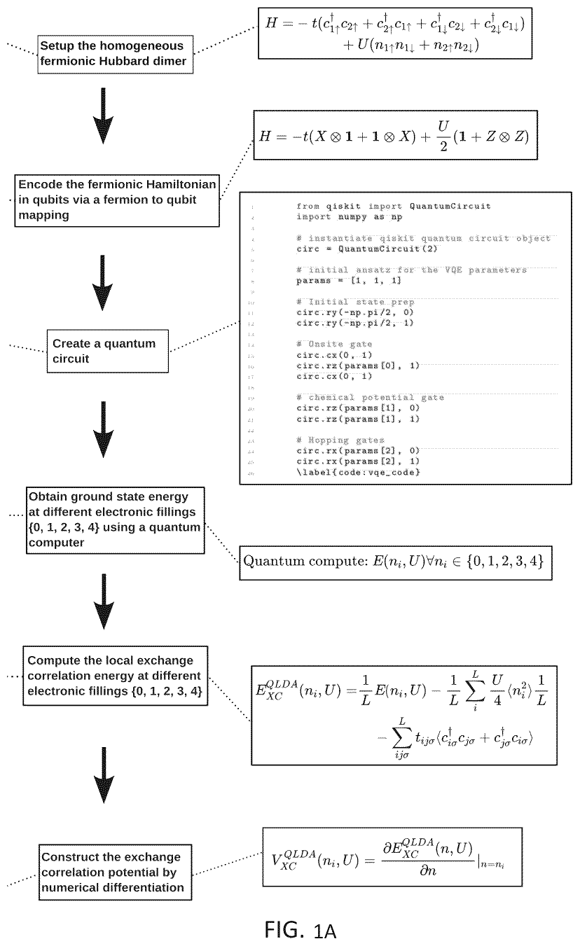

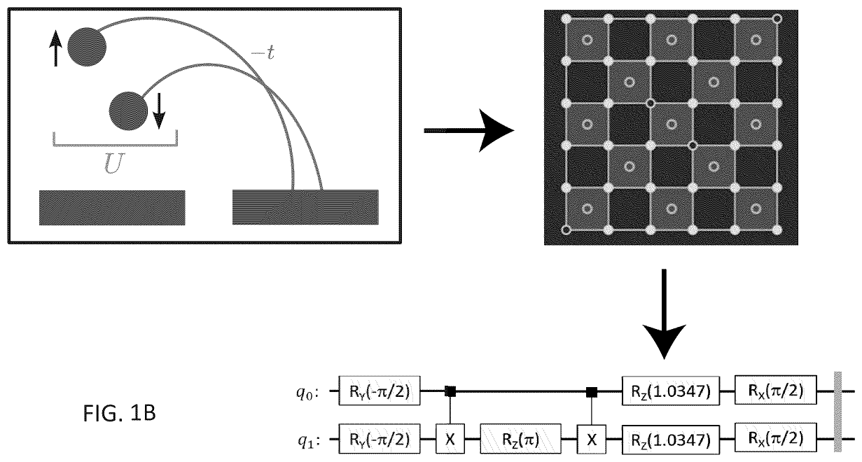

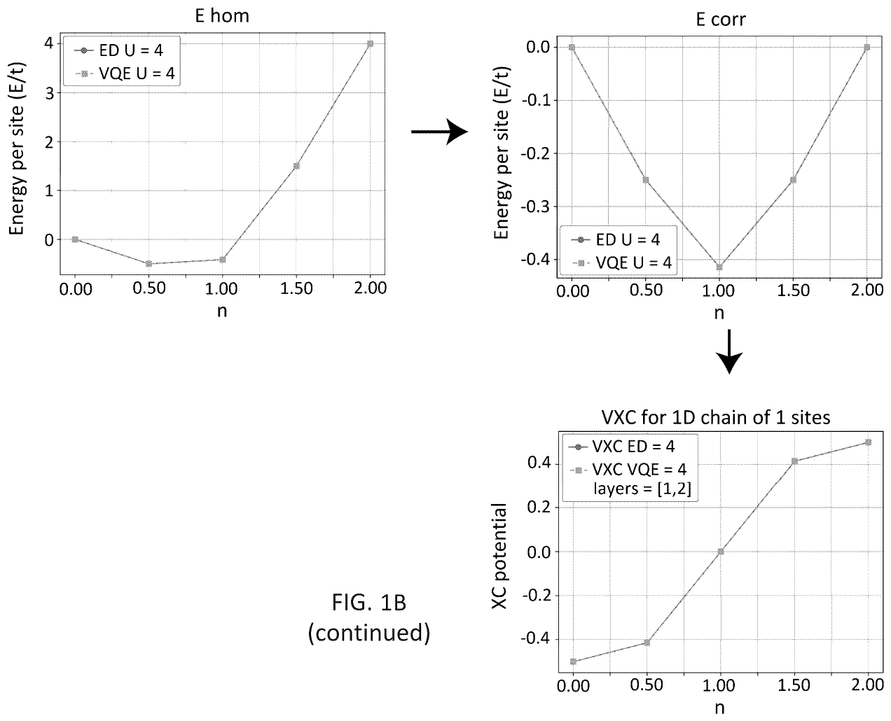

The Hubbard dimer is the simplest model to which this process can be applied. In FIGS. 1A and 1B we show the process within a specifically designed flow chart for the Hubbard dimer of how to compute the XC potential, while in FIGS. 2A and 2B we show how to incorporate that functional into a classical DFT loop for the dimer.

Taking the process step by step, we have that:
(a) The ground state energy E_{dimer} of the Hubbard dimer (the second Hamiltonian), eq. [1] is transformed from a complex fermionic representation to Pauli representation via a fermionic encoding, in this case we choose a Jordan Wigner, which in the compressed representation equals

$$H = - t ( X ⊗ 1 + 1 ⊗ X ) + \frac{U}{2} ( 1 + Z ⊗ Z ) \qquad [ 2 ]$$

where X,Z are Pauli matrices, which can be decomposed into native hardware gates on a quantum device, and 1 is the identity operator. These steps are illustrated in the first 3 panels of FIG. 1.
(b) Equation [2] is then solved and normalised by the number of sites using a quantum computer by executing a VQE circuit which can be defined through code similar to the circuit depicted in FIG. 1, for which we must choose an ansatz, in this case the HVA.

The quantum portion of VQE computation requires two important specifications: (1) a fermion-to-qubit mapping and (2) a variational ansatz which comprises an initial state and circuit. Each of these considerations is related to the generated control signals.

The encoding defines how fermion operators are mapped into qubit operators which manifest as gates (quantum operations) in the quantum circuit. In our discussions we have chosen to use the conventional Jordan Wigner mapping which is a standard fermion-to-qubit mapping and associates each fermionic mode with a qubit. For example, if we consider the Hubbard dimer and just the interaction term on the first lattice site, we have that the fermionic Hamiltonian transforms as

$$U \times {\hat{n}}_{1 ↑} {\hat{n}}_{1 ↓} \to \frac{U}{4} \times Z ⊗ Z ,$$

where Z is the Pauli Z matrix.

There are different possible VQE ansatze which may be used; in this example the popular Hamiltonian Variational Ansatz (HVA) for VQE simulations is chosen. For HVA, the initial state can be prepared from the U=0 ground state of the Hubbard model, which comprises of a layer of depth nₓn_{y}−1 Givens rotations acting on adjacent modes, and its decomposition into signals depends on the native gates available on the hardware device. nₓ; n_{y} are the system dimensions because here we consider 2d. After the initial state has been created in the circuit, a representation of the VQE circuit is created which consists of a number of parametrised quantum gates. We illustrate this by returning to the Hubbard dimer example and specifically analysing the gate decomposition of the

$$\frac{U}{4} \times Z ⊗ Z$$

term in the HVA circuit. The task that we need complete is to represent the following term in the circuit: e^{iθ×U/4×Z⊗Z} as a quantum gate, where θ is a VQE parameter which is initialised and updated using classical optimisers. This precisely probes how this term is translated into a control signal. Ultimately, this depends on the native gates of the device, so there is no general answer on how to do this. However, a realistic practical assumption may be that CNOT and RZ gates are native on the hardware device. If so, then any then the above e^{iθ×U/4×Z⊗Z} term can be decomposed into a sequence of CNOT and RZ gates. Moreover, this is also an operational way in which the aforementioned Givens rotations can be decomposed into signal controls for a device.

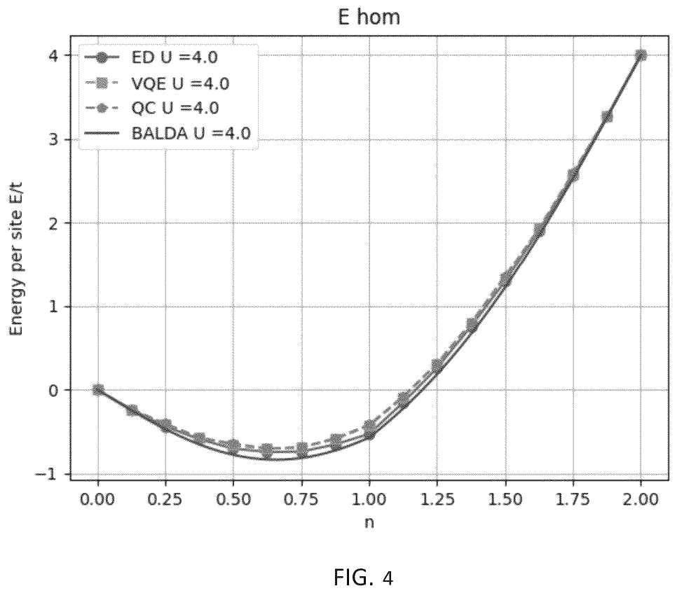

(c) The Hubbard dimer has 2 lattice sites, so it can at most have 4 fermions, which means the quantum computer must produce 4 data points, these 4 data points are plotted as a function of the fractional fermion filling, as highlighted in FIG. 1 within the fourth horizontal panel. We note here that for larger systems, the approach is the same, but the density mesh is denser, due the number of fillings allowed being greater. A result which emphasises this is presented in FIG. 4, which has 16 data points (representing a 1×8 system) instead.

In FIG. 4 we see that noisy QC data can fall within 1% of in particular parameter regimes of the Hubbard model. This is in contrast to the classical mean field results, which fall outside of this 1% interval.

(c) The Hubbard dimer has 2 lattice sites, so it can at most have 4 fermions, which means the quantum computer must produce 4 data points, these 4 data points are plotted as a function of the fractional fermion filling, as highlighted in FIG. 1 within the fourth horizontal panel. We note here that for larger systems, the approach is the same, but the density mesh is denser, due the number of fillings allowed being greater. A result which emphasises this is presented in FIG. 4, which has 16 data points (representing a 1×8 system) instead.

FIG. 4 noisy QC data can fall within 1% of in particular parameter regimes of the Hubbard model. This is in contrast to the classical mean field results, which fall outside of this 1% interval.

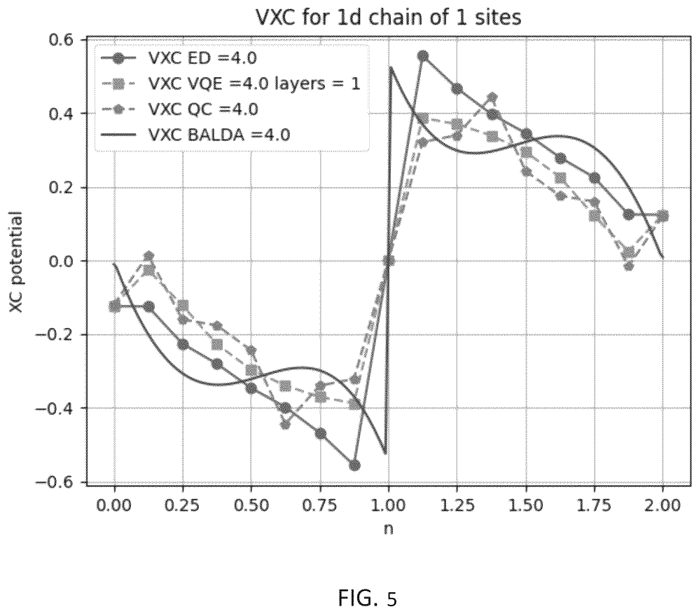

FIG. 5 shows the XC energy calculated from FIG. 4, and shows that this approach does reasonably well at capturing qualitative features of the XC potential as compared to exact classical methods (ED and BALDA).

Systematically, we see that for strongly correlated systems

$$( \frac{U}{t} > 1 )$$

the estimation of the density is also well described, as in FIG. 5.

(d) These 4 data points are then used to create the exchange correlation energy, as shown in the 5th panel of FIG. 1.

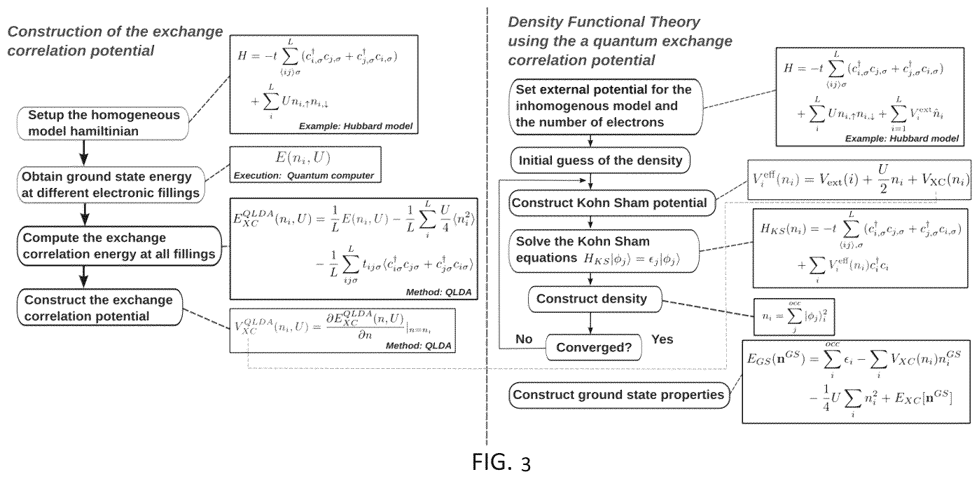

(e) Then the functional is created from these 4 points in the XC energy to give V_{XC}^{QLDA}(nᵢ, U), as shown in the 6th and final panel of FIG. 1, and further outlined in Section 6.2.1. This representation can be obtained through polynomial splining procedures, for example. This function is then stored and now can be used in a classical DFT loop, which remains the same as the right hand side of FIG. 3 for a specific choice of dimension and external potential Vₑₓₜ(i), i.e, the following set of Kohn Sham equations are solved for self consistently,

$${H}_{K S} ( {n}_{i} ) = - t {\sum }_{〈 ij 〉}^{L} ( {c}_{i}^{†} {c}_{j} + {c}_{j}^{†} {c}_{i} ) + {\sum }_{i} {V}_{i}^{e f f} ( {n}_{i} ) {c}_{i}^{†} {c}_{i} where \qquad [ 3 ]$$

$${H}_{K S} ( {n}_{i} ) = - t {\sum }_{〈 ij 〉}^{L} ( {c}_{i}^{†} {c}_{j} + {c}_{j}^{†} {c}_{i} ) + {\sum }_{i} {V}_{i}^{e f f} ( {n}_{i} ) {c}_{i}^{†} {c}_{i} and \qquad [ 4 ]$$

$$\frac{d F ( n )}{d {n}_{i}}$$

and is the unknown multivariate exchange correlation potential approximated to produce an approximation to the ground state energy and density.

(f) That is, in more detail, in the first panel of FIGS. 2A and 2B we start by setting an external potential, which in this case is only defined at two lattice points, i.e (v₁, v₂), as we are dealing with the dimer.

(g) Then an initial guess of the density is made, which is proportional to the external potential, as in the second panel of FIGS. 2A and 2B.

(h) Finally, the Kohn Sham equations are solved until self-consistency, as depicted in the rest of the panels in FIGS. 2A and 2B.

The Hubbard Dimer analysed within the Site Occupation Functional Theory (SOFT) formalism with Kohn Sham self consistency across a number of different approximations, including exact approaches (Exact Diagonalisation (ED)), mean field (Hartree Fock (HF)) and variational methods (Variational Quantum Eigensolver (VQE)). Moreover, we introduce two approaches that can compute the physical features of the universal functional, which is,

$$F [ n ] = min Ψ \to n ( Ψ \vert \hat{T} + \hat{V} \vert Ψ 〉 = T [ n ] + {V}_{e e} [ n ] = T [ n ] + {E}_{H} [ n ] + {E}_{X C} [ n ]$$

where T[n] is the kinetic energy functional, E_{H} [n] is the Hartree term and E_{XC}[n] is the exchange correlation energy.

The dimer is defined by,

$${H}_{dimer} = {H}_{hubb} + {\sum }_{i = 1}^{2} {v}_{i} {\hat{n}}_{i} where$$

$${H}_{dimer} = - t {\sum }_{i σ}^{1} ( {c}_{i , σ}^{†} {c}_{i + 1 , σ} + {c}_{i + 1 , σ}^{†} {c}_{i , σ} ) + {\sum }_{i = 1}^{2} U {n}_{i , ↑} {n}_{i , ↓} .$$

Now, we define the following variables and set N=2, so that: Δv=v₁−v₂, v₁+v₂=0, n₁+n₂=2 and Δn=n₁−n₂. In doing so we can show that

$$\begin{matrix}〈 {H}_{d i m e r} 〉 = 〈 {H}_{h u b b} 〉 + 2 {v}_{2} ( 1 - 〈 {\hat{n}}_{1} 〉 ) \\ = 〈 {H}_{h u b b} 〉 + Δ v ( 1 - ( 1 - Δ n / 2 ) ) \\ = 〈 {H}_{h u b b} 〉 + Δ v Δ n / 2\end{matrix}$$

We can rearrange this formula to obtain the expression for the Hubbard energy, or the energy solely associated with exchange and correlation of the problem, i.e.

$$〈 {H}_{h u b b} 〉 = 〈 {H}_{d i m e r} 〉 - Δ v Δ n / 2$$

These expressions enable us to formulate Kohn Sham theory. By restricting our study to the N=2 and S_{z}=0 sector of the Hubbard dimer, which has the following matrix representation

$$( \begin{matrix}Δ v + U & - t & - t & 0 \\ - t & 0 & 0 & - t \\ - t & 0 & 0 & - t \\ 0 & - t & - t & - Δ v + U\end{matrix} )$$

where we have used the following basis states

{|⬆⬇,0 [symbol] ,|⬆,⬇ [symbol] ,|⬇,⬆ [symbol] ,|0,⬆⬇ [symbol] }.

The VQE circuit used here is illustrated in Section 6.1 and is based on the compressed representation of the Hubbard dimer at half-filling and S_{z}=0 with additional single qubit gates to account for the external potential. We have created two subroutine algorithms that compute F(U/t, δn) for the Hubbard dimer: (i) qDFT-max and (ii) qDFT-min. Both algorithms utilise the ground state energy computed by VQE which then enters into an outer optimisation loop to find the exchange correlation functional.

The outer optimisation loop is executed as a quantum kernel that uses interpolative and extrapolative procedures that allow for continuous representations of the exchange correlation potential whilst reducing finite size effects. Precisely, the quantum kernel protocol is:
a. Obtain the exchange correlation potential using a quantum computer for system size L. This means executing at most 2L quantum computations resulting in a function

$${V}_{X C} ( n ) where n = \frac{1}{L} \{ 0 , … , L , … 2 L \}$$

the discrete fermion fillings.
b. Obtain V_{XC}(n) in a continuous representation of via a classical interpolative procedure, e.g cubic splines. (It is possible to impose constraints on the functional, for example if the fundamental gap, Δ=V_{XC}(1⁺)−V_{XC}(1⁻), is known a priori, it can impose two extra constraining points near

$$\frac{N}{L} = 1 .$$

It is possible to know this a priori from

$$Δ = U - {lim}_{n \to {1}^{-}} {\partial }_{n} {ϵ}_{GS} ( n < 1 ) .$$

c. Repeat step (a)-(b) for M different system sizes, for example in 2d we could have

$$L = \{ {( {n}_{x} , {n}_{y} )}_{1} , … , {( {n}_{x} , {n}_{y} )}_{M} \} = \{ ( 2 , 2 ) , ( 3 , 3 ) , … , ( M , M ) \} .$$

d. Extrapolate using L to the thermodynamic limit, if the scaling is possible, otherwise take V_{XC} (n) from the largest system size.
   Extrapolating to thermodynamic limit can alleviate finite size effects. In the context of the Bethe Ansatz solution to the Hubbard Model in one dimension ‘thermodynamic limit’ means the limit L→∞, N→∞, and N/7 is constant at zero temperature.

### Representative Results

### The Hubbard Dimer

### Quantum Hybrid Density Functional Theory

Density Functional Theory (DFT) is a very successful method for studying weakly correlated electronic systems.
It encounters numerous pitfalls for strongly correlated electronic systems, where it can often qualitatively fail in its predictions.

### Lattice Density Functional Theory (LDFT)

### The Inhomogeneous Hubbard Model is Given by

$$H = - t {\sum }_{〈 ij 〉 σ}^{L} ( {c}_{i , σ}^{†} {c}_{j , σ} + {c}_{j , σ}^{†} {c}_{i , σ} ) + {\sum }_{i}^{L} U {n}_{i , ↑} {n}_{i , ↓} + {\sum }_{i = 1}^{L} {v}_{i} {\hat{n}}_{i} ,$$

DFT Maps this Hamiltonian into a Single Particle One with an Effective Potential

$${H}_{KS} ( {n}_{i} ) = - t {\sum }_{〈 ij 〉}^{L} ( {c}_{i}^{†} {c}_{j} + {c}_{j}^{†} {c}_{i} ) + {\sum }_{i} {V}_{i}^{eff} ( {n}_{i} ) {c}_{i}^{†} {c}_{i} ,$$

$$where$$

$${V}_{i}^{eff} ( {n}_{i} ) = {V}_{ext} ( i ) + \frac{u}{2} {n}_{i} + \frac{dF ( n )}{{dn}_{i}}$$

$$where \frac{dF ( n )}{{dn}_{i}}$$

is the unknown exchange correlation potential. A current goal of this work is to approximate

$$\frac{dF ( n )}{{dn}_{i}}$$

with VQE.
The algorithm for LDFT is

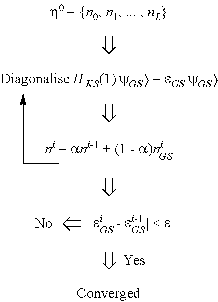

### The Hubbard Dimer in LDFT

$${H}_{dimer} = {H}_{hubb} + {\sum }_{i = 1}^{2} {v}_{i} {\hat{n}}_{i}$$

$$where$$

$${H}_{hubb} = - t {\sum }_{i σ}^{1} ( {c}_{i , σ}^{†} {c}_{i + 1 , σ} + {c}_{i + 1 , σ}^{†} {c}_{i , σ} ) + {\sum }_{i = 1}^{2} {Un}_{i , ↑} {n}_{i , ↓}$$

Now, we define the following variables and set N=2, so that: Δv=V₁−v₂, v₁+v₂=0, n₁+n₂=2 and Δn=n₁−n₂. In doing so we can show that

$$〈 {H}_{dimer} 〉 = 〈 {H}_{hubb} 〉 + 2 {v}_{2} ( 1 - 〈 {\hat{n}}_{1} 〉 ) = 〈 {H}_{hubb} 〉 + Δ v ( 1 - ( 1 - Δ n / 2 ) ) = 〈 {H}_{hubb} 〉 + Δ v Δ n / 2$$

We can rearrange this formulae to obtain the expression for the Hubbard energy, or the energy solely associated with exchange and correlation of the problem, i.e.

$$〈 {H}_{hubb} 〉 = 〈 {H}_{dimer} 〉 - Δ v Δ n / 2$$

These expressions enable us to formulate Kohn Sham theory. By restricting our study to the N=2 and S_{z}=0 sector of the Hubbard dimer, which has the following matrix representation

$$( \begin{matrix}Δ v + U & - t & - t & 0 \\ - t & 0 & 0 & - t \\ - t & 0 & 0 & - t \\ 0 & - t & - t & - Δ v + U\end{matrix} )$$

where we have used the following basis states

{|⬆⬇,0 [symbol] ,|⬆,⬇ [symbol] ,|⬇,⬆ [symbol] ,|0,⬆⬇ [symbol] }.

### Formulation of Certain Subroutines

formulated starting from the Hohenberg-Kohn variational principle which states that the total energy of the ground state is given by:

$$E [ {V}_{ext} ( r ) ] = {min}_{n ( r )} [ F [ ( r ) ] + \int {V}_{ext} ( r ) n ( r ) ]$$

We obtain the formulation of a first algorithm, qDFTmin, by isolating F[(r)] and solving it for the Hubbard dimer as a function of Vₑₓₜ(r). Therefore, we show that using a quantum computer and given an external potential it is possible to find the corresponding universal functional energy, from which the XC potential can be found.
On the other hand, we obtain a second algorithm, qDFTmax, from Lieb's formulation of DFT, which states that the universal functional can be obtained from maximising over the external potential for a given density, i.e.,

$$F [ n ( r ) ] = {max}_{{V}_{ext} ( r )} [ E [ {V}_{ext} ( r ) ] - \int {V}_{ext} ( r ) n ( r ) ] .$$

### The VQE Circuit

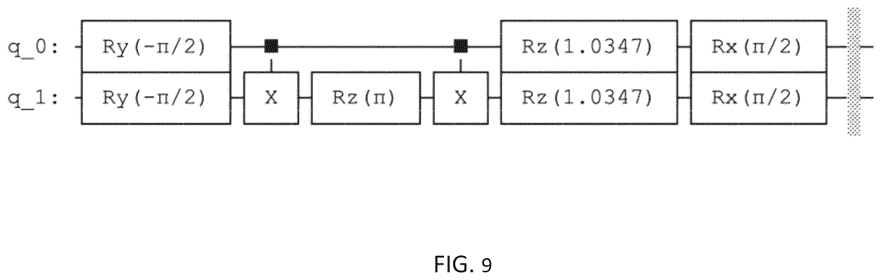

The VQE circuit is given in FIG. 9 in a compressed representation. This circuit can be tested and verified by running by running a suitable python code, for example Quskit modelling code:

| | |
|---|---|
| 1 | from qiskit import QuantumCircuit |
| 2 | import numpy as np |
| 3 | |
| 4 | # instantiate qiskit quantum circuit object |
| 5 | circ = QuantumCircuit (2) |
| 6 | |
| 7 | # initial ansatz for the VQE parameters |
| 8 | params = [1, 1, 1] |
| 9 | |
| 10 | # Initial state prep |
| 11 | circ .ry(−np.pi /2, 0) |
| 12 | circ .ry(−np.pi /2, 1) |
| 13 | |
| 14 | # Onsite gate |
| 15 | circ .cx (0, 1) |
| 16 | circ .rz( params [0] , 1) |
| 17 | circ .cx (0, 1) |
| 18 | |
| 19 | # chemical potential gate |
| 20 | circ .rz( params [1] , 0) |
| 21 | circ .rz( params [1] , 1) |
| 22 | |
| 23 | # Hopping gates |
| 24 | circ .rx( params [2] , 0) |
| 25 | circ .rx( params [2] , 1) |
| 26 | \ label { code : vqe_code } |

### RESULTS

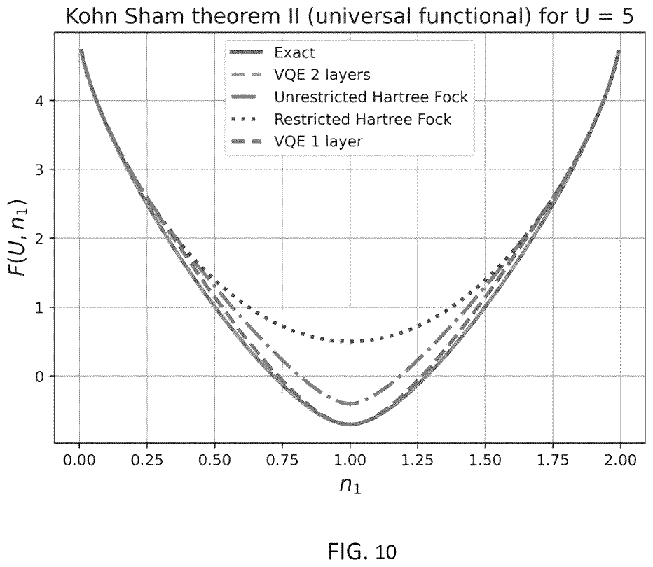

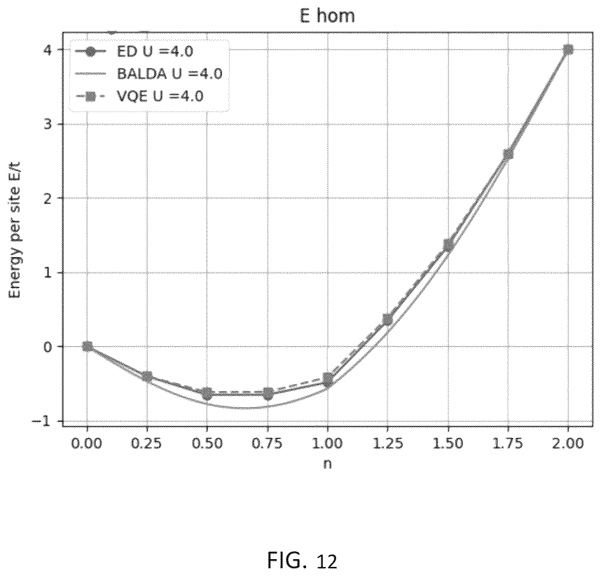

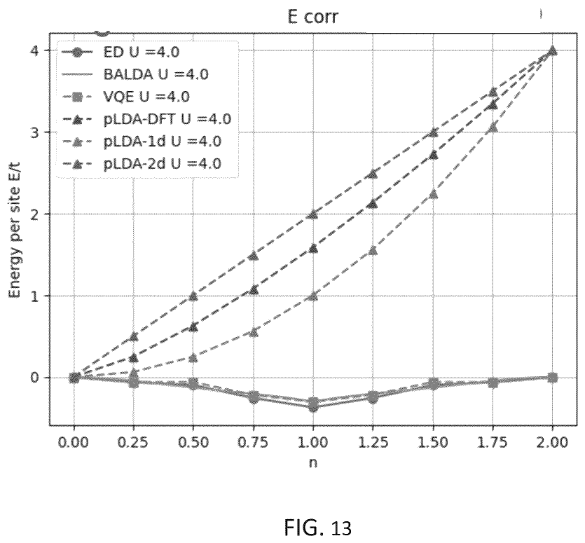

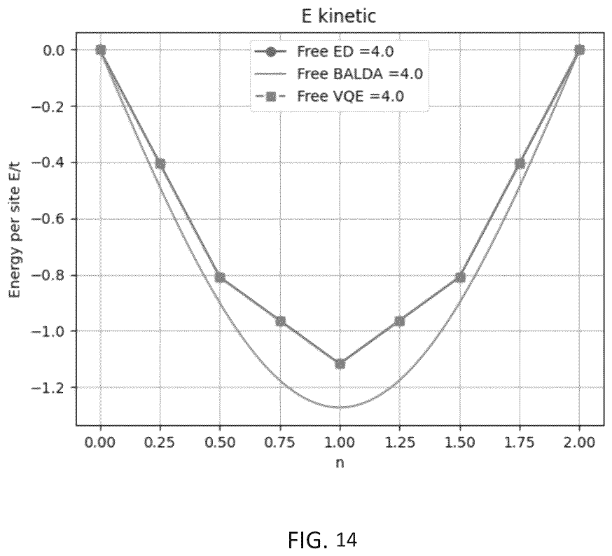

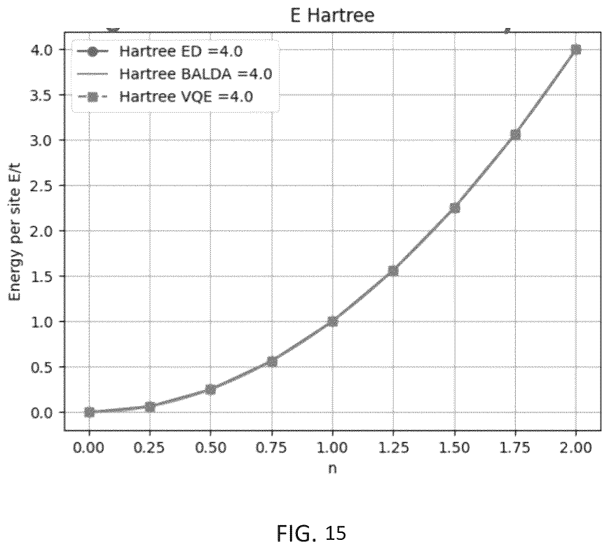

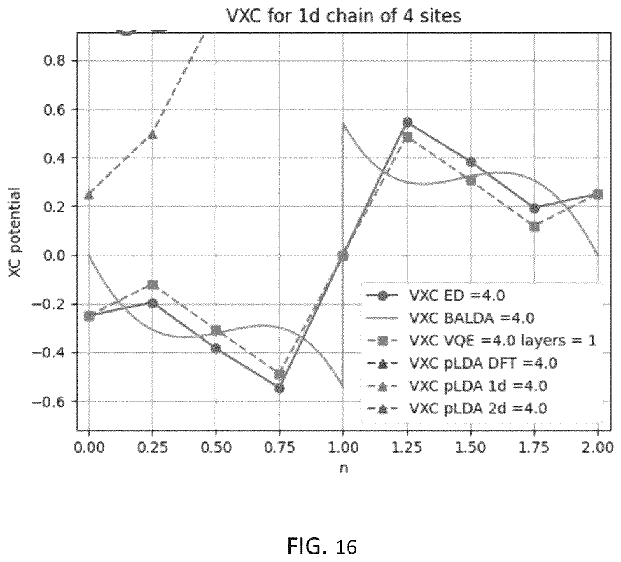

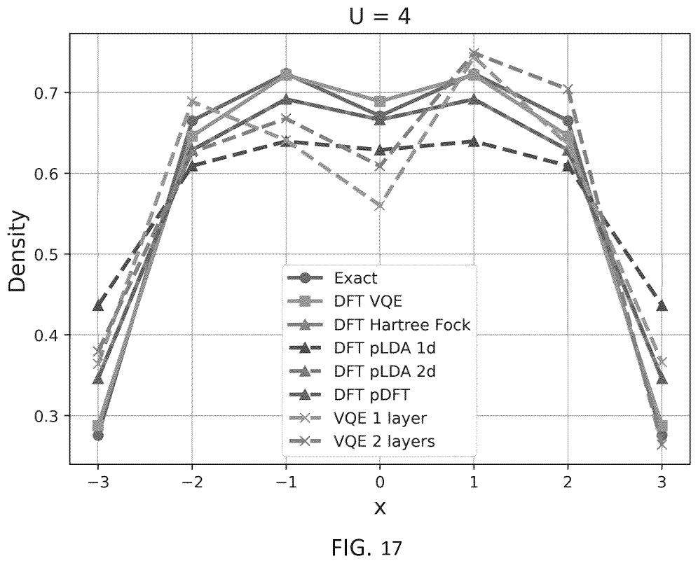

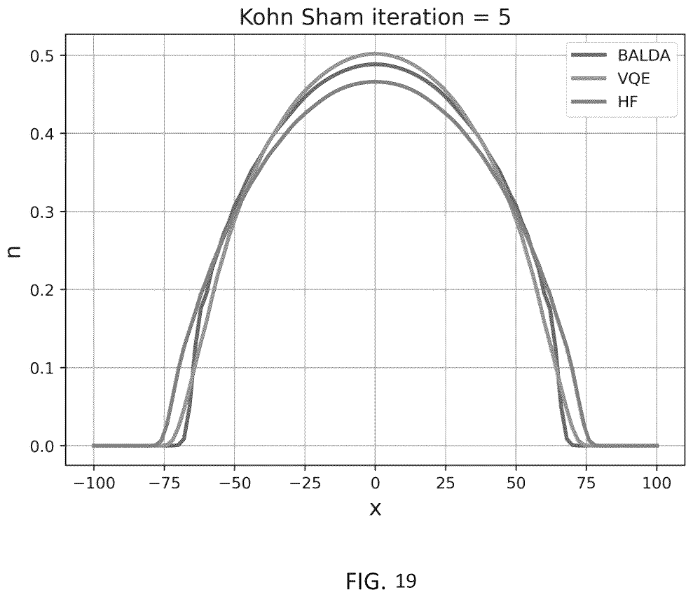

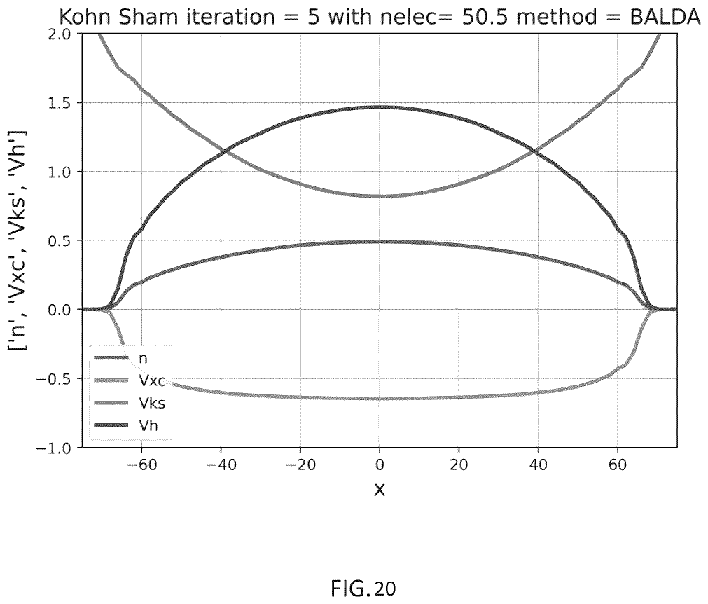

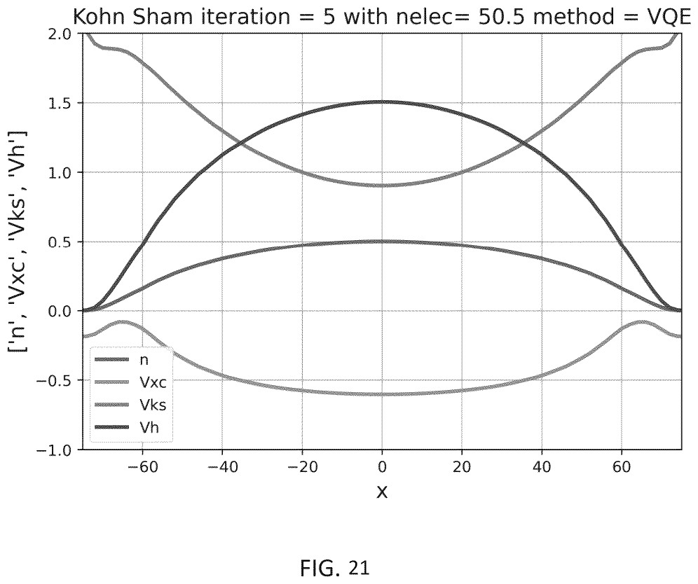

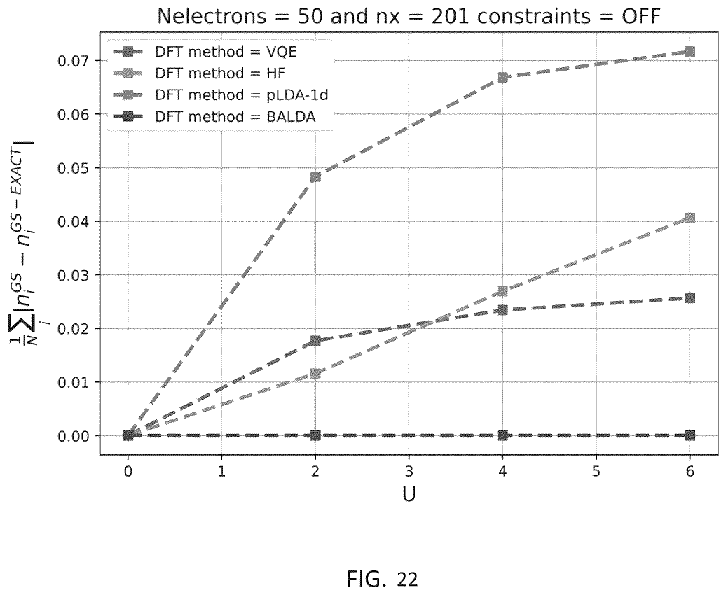

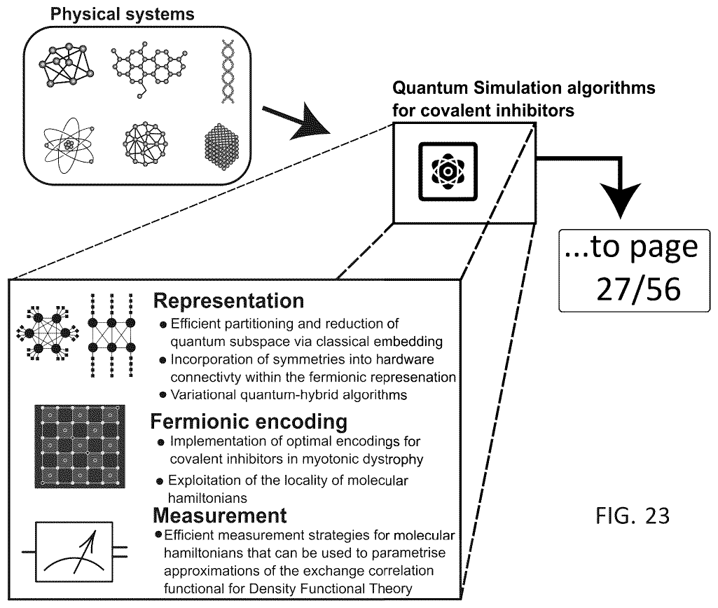

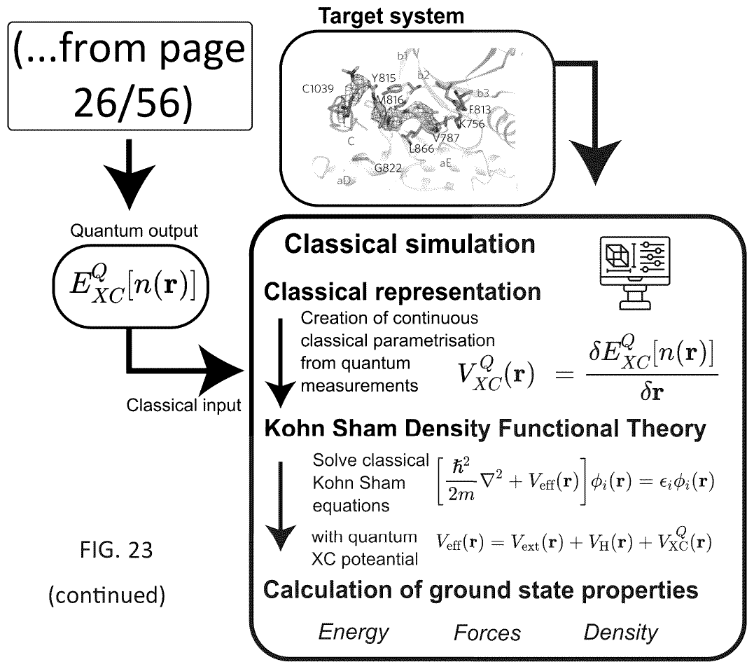

Various results of the process including comparisons against existing methods, are shown in FIGS. 10 to 22. A core result here is the improvement shown in the hybrid DFT methods set out herein show an unexpectedly beneficial result, despite the simplifications implemented to allow the problem to be tractable on modem NISQ hardware.

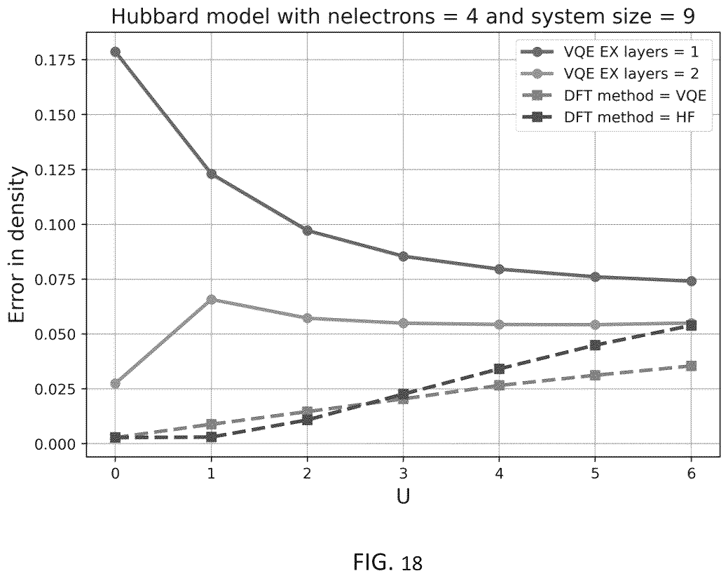

As one example, in FIG. 18, the DFT method (VQE line) outperforms all other methods (with the exception of Hartree Fock at low U values. In particular, it is substantially more accurate than the raw VQE algorithm of depth 1.

### Extending Beyond the Dimer

The dimer is extended by simply adding more sites, and results in the inhomogeneous Hubbard model (IHM) is given by,

$$H = - t {\sum }_{〈 ij 〉 σ}^{L} ( {c}_{i , σ}^{†} {c}_{j , σ} + {c}_{j , σ}^{†} {c}_{i , σ} ) + {\sum }_{i}^{L} {Un}_{i , ↑} {n}_{i , ↓} + {\sum }_{i = 1}^{L} {V}_{ext} ( i ) {\hat{n}}_{i} ,$$

where Vₑₓₜ(i) is the external lattice potential, U is the interaction strength, t is the hopping parameter and L is the system size. In 1 dimension L=nₓ where nₓ is the number of sites of the lattice along the x-axis; in 2 dimensions L=nₓn_{y}, and so on.

The Hamiltonian Variational Ansatz for inhomogeneous systems takes account of the densities which appear attached to the external potential in the above equation. These manifest as additional single particle density terms in the Hamiltonian due to the presence of the external potential, that would not be there if one wasn't constraining it to specific values of the external potential, and consequently specific values of density observables due to the Hohenberg-Kohn theorems.

DFT maps into the effective Kohn-Sham system, where the many body interaction term is absorbed into a one-body potential Vᵢ^{eff},

$${H}_{KS} ( {n}_{i} ) = - t {\sum }_{〈 ij 〉}^{L} ( {c}_{i}^{†} {c}_{j} + {c}_{j}^{†} {c}_{i} ) + {\sum }_{i} {V}_{i}^{eff} ( {n}_{i} ) {c}_{i}^{†} {c}_{i} ,$$

$$where$$

$${V}_{i}^{eff} ( {n}_{i} ) = {V}_{ext} ( i ) + \frac{U}{2} {n}_{i} + \frac{dF ( n )}{{dn}_{i}}$$

$$\frac{dF ( n )}{{dn}_{i}}$$

is the unknown multivariate exchange correlation potential the central object of interest. Once

$$\frac{dF ( n )}{{dn}_{i}}$$

is approximated, the self-consistent Kohn-Sham scheme is initiated and the primary parts of the classical algorithm are highlighted in Section 6.1. Now we introduce quantum techniques that compute F(n).

### The Quantum Local Density Approximation

The Quantum Local Density Approximation (QLDA) expresses the form of the XC energy functional in a compact representation. Starting with the general functional form of the object, we have,

$${E}_{XC} = {E}_{XC} ( {n}_{1} , … , {n}_{L} , U )$$

QLDA approximation to the XC energy functional then is,

$${E}_{XC}^{QLDA} ( {n}_{i} , U ) = \frac{1}{L} {E}_{hom}^{VQE} ( {n}_{i} , U ) - \frac{1}{L} {\sum }_{ij σ}^{L} {t}_{ij σ} ( {c}_{i σ}^{†} {c}_{j σ} + {c}_{j σ}^{†} {c}_{i σ} 〉 - \frac{1}{L} {\sum }_{i}^{L} \frac{U}{4} 〈 {n}_{i}^{2} 〉$$

where Eₕₒₘ^{VQE}(nᵢ, U) is the energy of the homogeneous Hubbard model, with Vₑₓₜ(i)=0, computed using VQE of a specified depth and occupation number. In comparison to E_{XC}, above, the total energy has been assumed to be a sum of the average energy in a homogeneous Hubbard chain of size L. The protocol for obtaining this function is sketched in Section 6.2, and once it is known the XC potential can be computed by differentiation, i.e.

$${V}_{XC}^{QLDA} ( {n}_{i} , U ) = \frac{\partial {E}_{XC}^{QLDA} ( n , U )}{\partial n} {\vert}_{n = {n}_{i}}$$

Thus, for a system of size L the maximum number of VQE calls to approximate E_{XC} is given by 2L. By constructing interpolative and extrapolative techniques, which are briefly touched upon in Section 6.3, it is possible to modify the number of these calls, whose upper bound will be 2ΠᵢLᵢ, where Lᵢ is the set containing the number of system sizes considered, e.g. L={4, 5, 6} would require at most 240 quantum computations to be performed. At convergence, the ground state energy is determined by

$${E}_{GS} ( {n}^{GS} , U ) = {\sum }_{i}^{occ} {ε ( U )}_{i}^{KS} - {\sum }_{i} {V}^{XC} ( {n}_{i} , U ) {n}_{i}^{GS} - \frac{1}{4} U {\sum }_{i} {n}_{i}^{2} + {E}_{XC}^{QLDA - TOT} [ {n}^{GS} , U ]$$

where εᵢ^{KS} are the eigenvalues of the occupied Kohn-Sham system and the ground state density is determined by

$${n}_{i}^{GS} = {\sum }_{j}^{occ} {ϕ}_{ij}^{2}$$

where ϕᵢⱼ is the i′th element of the j′th occupied Kohn Sham eigenvector. The total exchange correlation energy is given by,

$${E}_{XC}^{QLDA - TOT} [ {n}^{GS} , U ] = {\sum }_{i} {E}_{XC}^{QLDA} ( {n}_{i} , U ) .$$

Here, multivariate means that:

$${E}_{XC} = {E}_{XC} ( {n}_{1} , … , {n}_{L} , U )$$

where E_{XC} (n₁, . . . , n_{L}, U). is a functional of the density at L sites, i.e it is a function of L+1 variables. The function depends on L+1 variables in potentially a nonlinear way. The local density approximation assumes that the function can be separated linearly, such that

$${E}_{XC}^{QLDA - TOT} [ {n}^{GS} , U ] = {\sum }_{i} {E}_{XC}^{QLDA} ( {n}_{i} , U )$$

i.e the function is a sum of L terms in a linear way. Including non locality means that the function would depend not only on density at site i, but also at additional sites, for example in the nearest neighbour case in one dimension it would mean,

$${E}_{XC}^{QLDA - TOT} [ {n}^{GS} , U ] = {\sum }_{i} {E}_{XC}^{QLDA} ( {n}_{i - 1} , {n}_{i} , {n}_{i + 1} , U ) .$$

In a continuous sense, it just means that the functional not only depends on the density at site i, but also on the derivative, i.e.

$${E}_{XC}^{QLDA - TOT} [ {n}^{GS} , U ] = {\sum }_{i} {E}_{XC}^{QLDA} ( {n}_{i} , {\partial }_{x} {n}_{i} , U )$$

if we are in 1 dimension. In the classical DFT literature this approach is known as the Generalised Gradient Approximation (GGA).

The true XC functional is multivariate and a complicated function of the entire density at all points in space. Knowing exactly the multivariate functional representation for a given model would provide the exact solution for the ground state properties for the homogeneous and all inhomogeneous variations of that model by only solving the classical Kohn-Sham equations, which scale as L³, where L is the system size.

Theoretically this is NP-hard, as it necessitates the solution of the exact homogeneous problem, for example the Hubbard model. However, with a near term quantum computer it may be possible to qualitatively capture important features of the multivariate functional with low-depth and low-quality quantum simulations, which lead to better solutions when this is fed into the DFT loop than classical approximations to the functional can achieve. Practically, the external potential of the inhomogeneous model needs to be varied, in a more general way than it is done in the qDFT-max procedure as implemented for the Hubbard dimer.

Nonlocality can be incorporated into this approximate functional by making the XC energy depend not only on the local density at each site, but also on its derivative. This follows the spirit of where in practical ab initio DFT calculations a very successful approach that supplements ideas based on local density approximations are generalised gradient approximations.

In FIGS. 1A to 2B we present the detailed steps involved in performing a full QDFT computation for the Hubbard dimer. Specifically, when this process is extended to more complicated scenarios we distinguish these by (1) Hubbard models of larger dimension, as is highlighted generically in FIG. 1, or (2) simulate materials/chemical systems. In the case of (1), we remind ourselves that L=nₓ×n_{y}, where nₓ=1 and n_{y}=2 for the Hubbard dimer. nₑ=2L, i.e the maximum number of fermions in the system. Generally, the number of energy data computed by a quantum computer in a system of size L is 2L and they are plotted in the same way as FIG. 5a. This is precisely the main operational difference between running the Hubbard dimer and a system size of larger dimension. For more realistic systems that can be used for materials and/or chemical simulation the general strategies are outlined in Section 2. Importantly, what needs adaptation here is the connectivity of the underlying lattice model, the values of the parameters which are used to the define the model, and the form of the external potential studied. The type of encoding and VQE ansatz used may also have to be modified, depending on the chosen system of interest. In principle, any systems which have a representation of the form in the “Direct parameterisation of tight binding models” section below, these include for example semiconductors, superconductors, battery materials, photovoltaics, and thermoelectric materials, to name only a few. In specific reference to the plots, the primary difference between the approach for the dimer and more general systems essentially occurs in FIG. 1 fourth panel, where the ground state energy at different fillings is computed, where for more general systems the density mesh is denser, as in FIG. 5a.

It is also worth highlighting that in the weakly correlated regime that classical mean field theory (Hartree-Fock) can be used as a more accurate solver, as can be seen in FIGS. 5A and 5B.

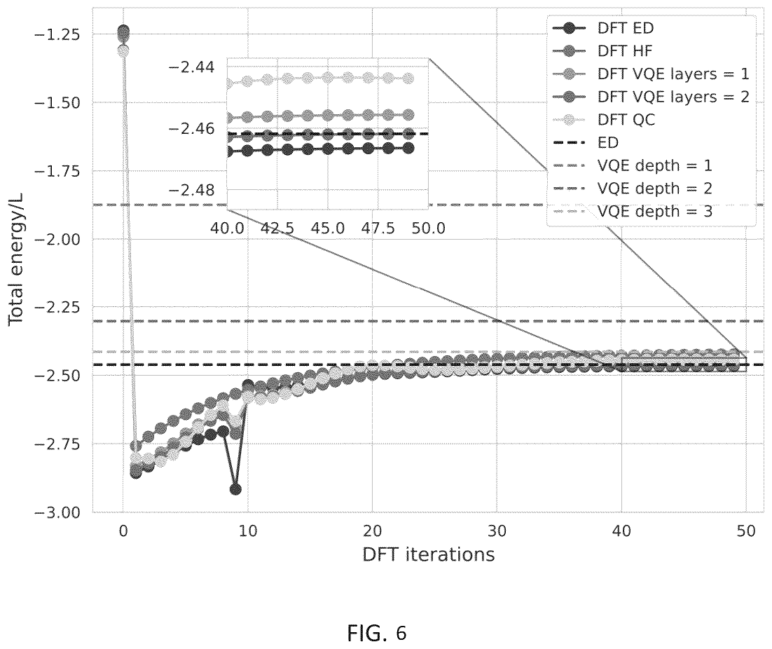

In FIG. 1 and FIG. 2 we present the detailed steps involved in performing a full QDFT computation for the Hubbard dimer. We have commented in the discussion after the Hubbard dimer walkthrough how to generalize this approach to more complicated systems, in particular also materials simulations. In specific reference to the plots, the primary difference between the approach for the dimer and more general systems essentially occurs in FIG. 1 fourth panel, where the ground state energy at different fillings is computed, where for more general systems the density mesh is denser, as in FIG. 5a.
A quantum computer was used to obtain the energies of the homogeneous Hubbard model using VQE, as highlighted in left part of FIG. 3. The result of this is a discrete function, such as in FIG. 5b
This data is then used to construct the exchange correlation potential, shown in FIG. 5b.
Finally the XC potential in FIG. 5b is queried 50 times inside a DFT loop for an inhomogeneous model (see FIG. 6) with a random external potential (a test case) and illustrates then convergence of the ground state energy from various methods compared to the exact answer as computed using Exact Diagonalisation.

To help this discussion, we outline where the quantum computation occurs in the overall algorithm, which is sketched in FIG. 3. A particular instance of the qDFT implementation works as follows:
Using a quantum computer, approximate (possibly to very low accuracy) the ground state energies as a function of electron filling for a chosen homogeneous interacting model. This is the point where the quantum computer is called and uses an algorithm to compute the ground state energy. For this instance it is the only part of the whole method which is performed by a quantum computer. However, for the numerics which we have performed to test and validate our methods, we have used the Varitional Quantum Eigensolver (VQE) and its Hamiltonian Varitional Ansatz (HVA) as the quantum algorithm of choice. This is illustrated in the left part of FIG. 3.
The call to the quantum computer necessitates a quantum algorithm to approximate the ground state energy of a system. This approximation, however, does not necessarily need to be very accurate, as it is not used as the final output but fed into the DFT loop in order to steer the DFT algorithm towards a more accurate solution (see discussion, below, on intuition about why the combination of classical DFT with VQE works better). Therefore, any quantum algorithm that is capable of approximating the ground state energy—even crude approximations such as the very low-depth VQE achievable on current, noisy quantum hardware—can be used. We anticipate that better approximations would be advantageous when these are available, but we have demonstrated that our hybrid algorithm still produces very good solutions even when only low accuracy approximations are available from the quantum part of the method. For example, in the case of the Hubbard model we use VQE and the HVA and this determines how the circuit is designed. This is a common choice for computing the ground state energy of the Hubbard model.
Once the energy of the homogeneous model is computed, the exchange correlation energy is computed. In FIG. 3 we show how this is achieved within the Local Density Approximation of DFT for the homogeneous Hubbard model. We emphasise that this approximation is not strictly necessary but is used to demonstrate practically how to obtain the exchange correlation energy.
The exchange correlation energy is then differentiated to produce the exchange correlation potential.
This potential is then used inside the classical DFT algorithm which is entirely run on a classical computer, which is illustrated on the right part of FIG. 3

### Direct Parametrisation of Tight Binding Models:

Materials/Molecular Hamiltonians have the Following Representation

$$H = {\sum }_{σ} {\sum }_{{λ}_{1} , {λ}_{2}} {t}_{{λ}_{1} , {λ}_{2}} {c}_{{λ}_{1} , σ}^{†} {c}_{{λ}_{2} , σ} + {\sum }_{σ , {σ}^{′}} {\sum }_{{λ}_{1} , {λ}_{2} , {λ}_{3} , {λ}_{4}} {V}_{{λ}_{1} {λ}_{2} {λ}_{3} {λ}_{4}}^{Coulomb} {c}_{{λ}_{1} , σ}^{†} {c}_{{λ}_{2} , {σ}^{′}}^{†} {c}_{{λ}_{3} , {σ}^{′}} {c}_{{λ}_{4} , σ} + {\sum }_{{λ}_{1}} {V}_{{λ}_{1}}^{ext} ( {c}_{{λ}_{1} , ↑}^{†} {c}_{{λ}_{1} , ↑} + {c}_{{λ}_{1} , ↓}^{†} {c}_{{λ}_{1} , ↓} ) ,$$

where λ is the site or orbital index and represents the quantum numbers of the fermions, excluding spin which is explicitly labelled. V_{λ}_{₁}_{λ}_{₂}_{λ}_{₃}_{λ}_{₄}^{Coulomb} is the four index Coulomb tensor, V_{λ1}^{ext} is the single index external potential, and t_{λ}_{₁}_{,λ}_{₂} is the two-index pure hopping terms is an off diagonal matrix. V_{λ}_{₁}^{ext} is set by the material or molecule under examination, while the other parameters can be computed from Ab Initio calculations. Once the homogeneous Hamiltonian is known (through setting V_{λ}_{₁}^{ext}=0), its local exchange correlation energy can be computed similarly as described in FIG. 3 using quantum algorithms. The obtained functional can then be used for larger system sizes with inhomogeneities. We note that depending on the material of choice, the Hamiltonian defined above is general, so that 1d, 2d, and 3d systems can be described; as well as systems with long range hoppings, interactions (e.g. a multiorbital Slater-Kanamori system) and varying connectivities. In principle, every material that has a unique homogeneous Hamiltonian would also have a unique XC potential.

Similar approaches, i.e. extending lattice models to continuous systems, have been applied within classical algorithms, using different approximate functionals, the first application of DFT within the lattice context to continuous systems. In these works, well known values for t_{λ}_{₁}_{, λ}_{₂} are used to model the hopping processes, while V_{λ}_{₁}_{λ}_{₂}_{λ}_{₃}_{λ}_{₄}^{Coulomb} is approximated to an onsite Hubbard interaction. We also note that it is also possible to truncate the above Hamiltonian to a subset of preferential active states within the Hilbert space, as described in. The advantage of doing so can dramatically reduces the quantum algorithm resource requirements. Technically, in this approach there emerges the well-known issues of incorporating double corrections.

### Direct Representation for Periodic Systems in a Continuous Basis:

In this case, we first define the ground state energy in terms of the continuous density n(r)

$$E [ n ] = T [ n ] + H [ n ] + {E}_{XC} [ n ] + \int n ( r ) {V}_{ext} ( r )$$

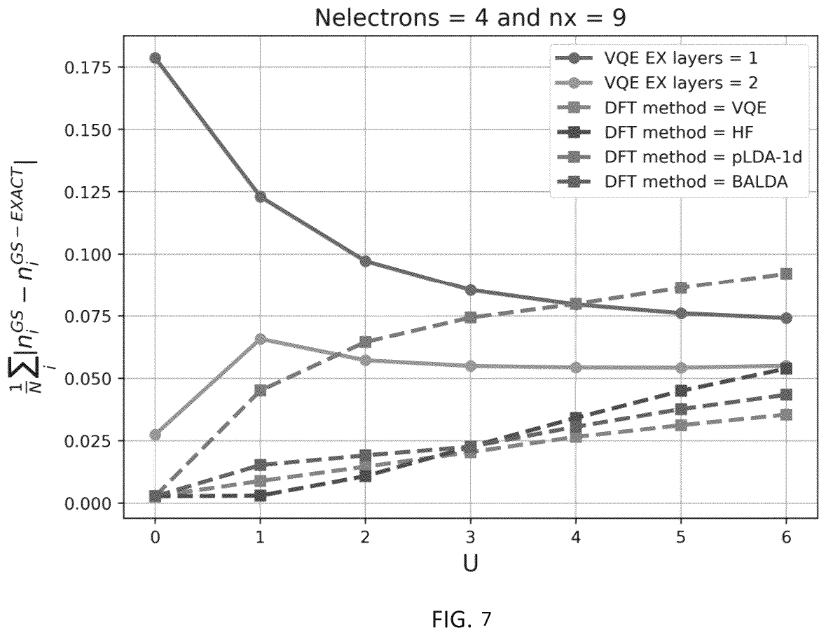

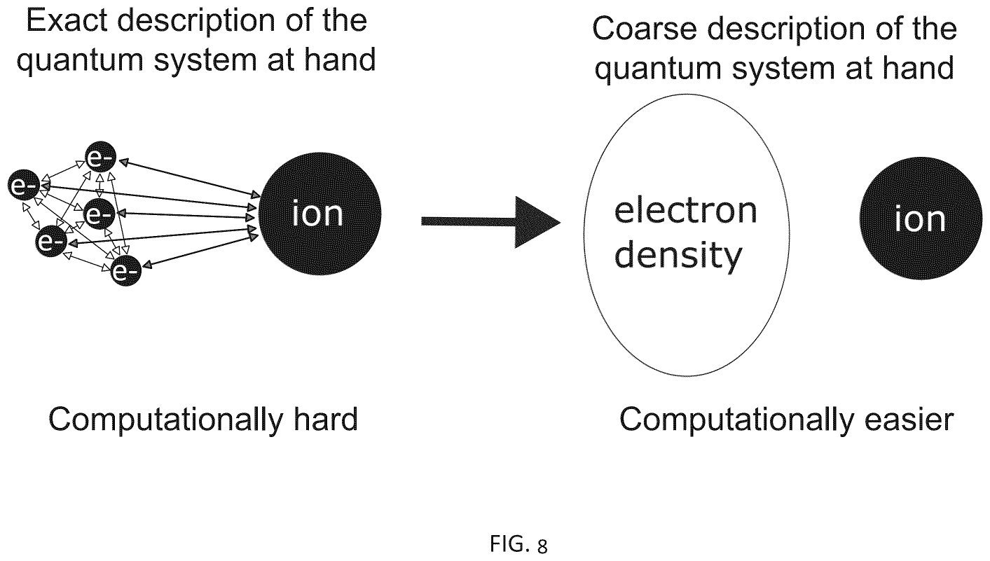

The basis dependent (e.g plane-wave, localised) Kohn Sham equations are

$$( - \frac{{∇}^{2}}{2} + {V}_{ext} ( r ) + {V}_{H} ( r ) + {V}_{XC} ( r ) ) {ϕ}_{j} ( r ) = {ϵ}_{j} {ϕ}_{j} ( r )$$

where Vₑₓₜ(r) is the external potential, V_{H} (r) is the Hartree potential, V_{XC}(r) is the exchange correlation potential, which needs to be approximated, and {∈ⱼ, ϕⱼ} are the Kohn-Sham eigenvalues and eigenvectors. Here we note the difference to the formulation in FIG. 7, which is discrete. The external potential in this case is defined by:

$${V}_{ext} ( r ) = {\sum }_{I} \frac{{Z}_{I}}{\vert {R}_{I} - r \vert} .$$

In this approach to solve the equation, the approximation to V_{XC}(r) originates in using a quantum algorithm to compute

$${E}_{XC} [ n ] = {E}_{GS} [ n ] - ( T [ n ] + H [ n ] + \int n ( r ) {V}_{ext} ( r ) ) ,$$

where E_{GS}[n] is computed on a quantum computer, for a given number of electrons and a Hamiltonian of the form above. Note that we have that for a given system we have subtracted away the contribution from the exchange correlation energy.

As a subroutine, the Variational Quantum Eigensolver algorithm is used to compute the exact exchange correlation energy contribution of the constituent fermions in the input molecular system. Importantly, for the application of myotonic dystrophy, we also incorporate into the algorithm how best to represent the input molecular Hamiltonians in order to minimize the number of quantum resources required to efficiently simulate the full target system.

By using a quantum computer to determine the unknown classical XC functional, the effects of exact exchange and correlation are automatically incorporated, a well-known shortfall of classical functional approximations that results in inaccurate ground state property calculations, most notably the over delocalization of electrons for a number of well-known molecular and periodic systems. We also note the benefit this approach has in terms of scalability over direct quantum mechanical treatments, such as classical configuration interaction approaches that suffer from the exponential scaling of the Hilbert space, or the direct implementation of quantum algorithms for the complete target system; here, once the functional is stored classically it can be used for any target system within the classical Kohn Sham framework, and agnostically incorporated with classical software packages, which scales polynomially in the system size and is an efficient classical algorithm.

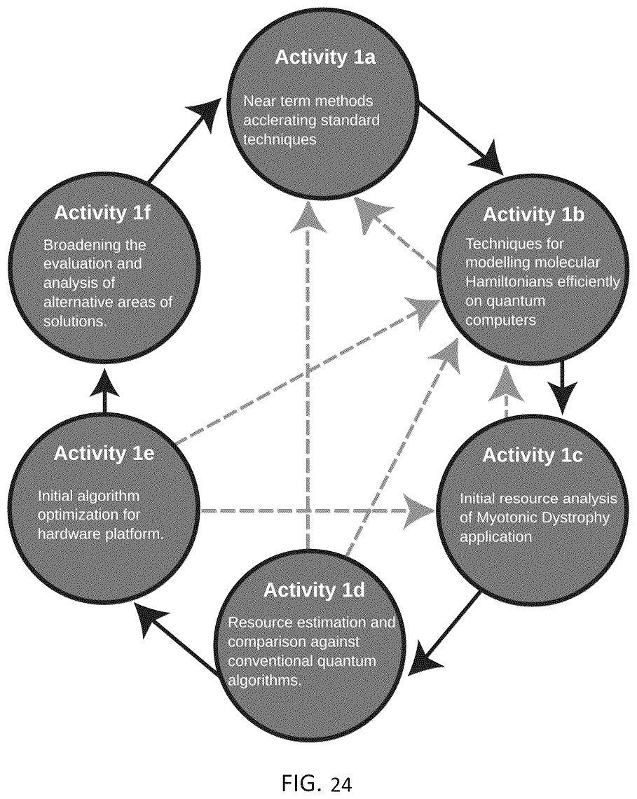

Recent work by the inventors discussed publicly here for the first time has shown that high fidelity XC potentials can be obtained from low depth, low quality VQE computations on quantum processors for the prototypical Fermi-Hubbard lattice model. The XC potentials can then be used inside a classical DFT loop executed on target systems which need not be the input system. Our results indicate that this approach can be an effective way of constructing XC potentials that include the effects of exchange and correlation, overcoming salient issues with classical functional designs, while simultaneously taking advantage of low depth and compact quantum circuits which can be run on NISQ devices. Here we build on these results by extending this formalism to fermionic Hamiltonians that describe the molecular systems relevant to this proposal. To do this, the following procedure is followed (with reference to FIG. 24):
Identify the minimal representations of fermionic Hamiltonians for myotonic dystrophy applications via classical simulation (optimizing the representation is related to Activity 1b). We note that the input systems need not be the eventual target system, but are used as proxies to generate high quality approximations to the XC energy and potential that are used to simulate the ground state properties of the target system (related to Activity 1c).
Construct quantum circuits that measure the XC energy from which the XC potentials are generated and permanently stored.
Extend the implementation of the algorithm presented in FIG. 1 to deal with molecular Hamiltonians. This includes providing an interface to classical ab initio software, which builds upon existing software suites developed internally for materials simulation.

Activity 1b: Techniques for modelling molecular Hamiltonians efficiently on quantum computers

It is imperative to map the classical representation of the molecular Hamiltonians into quantum circuits which can fit on the resources afforded by near term quantum devices. We use recent advancements in compact representations via embedding techniques, such as Density Matrix Embedding Theory (DMET), for molecular systems to reduce the number of qubits required in the quantum simulation. In these approaches, the classical molecular Hamiltonian is partitioned into two regions: one which is classically efficient and the other which uses direct quantum treatment. To reduce the gate depth, allowing for more accurate quantum computations of the XC energy, the connectivity of the parent molecular Hamiltonians can be optimized within the DMET approach by considering novel fragment to both mappings based on the hardware layout, a constraint which is not considered in purely classical approaches. Moreover, efficient fermionic encodings that exploit molecular locality and measurement strategies are needed to further reduce this depth. All of the above considerations have been extensively investigated by the inventors and discussed publicly here for the first time in recent works and we incorporate these optimisations into the overall framework as shown in FIG. 1 and implement the extensions of the DMET code needed to model molecular Hamiltonians, specifically tailoring their features to myotonic dystrophy inhibitor molecules.

### Activity 1c: Initial Resource Analysis of Myotonic Dystrophy Application.

In this work package we determine the quantum computing resources required to implement the algorithms developed in activities 1a/1b. Important resources to consider here are the number of qubits used, the number of 2-qubit quantum gates, and the quantum circuit depth. Recent work by the inventors discussed publicly here for the first time has developed a comprehensive resource estimation toolkit in the context of materials modelling, which enables the user to input a material's atomic structure, and outputs the quantum complexity of modelling that material via VQE or time-dynamics simulation. Here we perform a similar analysis for “standard” quantum algorithmic approaches in the context of myotonic dystrophy. In particular, we explore the fundamental physical representations of the fermionic systems involved in the myotonic dystrophy process, e.g. the importance of the chosen active space and how that relates to the computation of ground state properties. This activity requires the development of novel software tools as well as underpinning theory and numerical work. We additionally compare against the resources used by classical methods for addressing the relevant systems for myotonic dystrophy.

### Activity 1d: Resource Estimation and Comparison Against Conventional Quantum Algorithms.

This activity follows on from 1c by estimating resources used by the novel quantum algorithms developed in this project. Resource estimations for the quantum algorithm presented in FIG. 1 are made against conventional quantum algorithms for predicting ground state properties of the target system, such as direct application of the VQE. Our metric of choice for these estimates is the circuit depth and number qubits required to predict the ground state properties of the different target systems under consideration, before and after the optimisations from the above activities are considered.

We show how a collection of connected techniques, from local molecular embeddings, compact fermion encodings, native connectivity layouts of the parent Hamiltonians, to efficient measurement strategies, can impact the requirements needed to perform a NISQ computation for the application of myotonic dystrophy. Understanding the resource requirements for our novel approaches again involves a combination of theory, numerical work, and software development.

### Activity 1e: Initial Algorithm Optimization for Hardware Platform.

We perform an initial study of how the algorithm in FIG. 1 can be implemented and optimised. This platform has a number of features which make it well-suited to the proposed application domain, such as the ability to rearrange qubits and hence achieve effective all-to-all connectivity. In this activity, we determine the most efficient approach to represent and implement our algorithms to take advantage of these features. For example, this includes decomposing the operations that need to be performed in VQE in terms of hardware-native operations. As part of this, we undertake a finer-grained resource analysis that uses an accurate representation of the hardware's properties to determine our algorithms' true complexity and the level of performance that can be expected in later phases.

### Activity 1f: Broadening the Evaluation and Analysis of Alternative Areas of Solutions.

The algorithm presented in FIG. 1 can also be applied agnostically to other target molecular systems; this is especially true regarding the generated XC functionals and the underlying relationship between the parent molecular Hamiltonians and their encodings with hardware layouts. We explore the possibility of recycling these fully quantum XC potentials for other relevant human healthcare applications, which are potentially even higher-value than myotonic dystrophy. We also explore how quantum XC potentials can be incorporated into other quantum algorithms, such as time dynamical simulations that can probe excited state molecular properties.

### Activity 3: Development of Techniques for QM/MM Integration

The majority of QM/MM simulations use DFT for the QM part of the computation, primarily due to their computational cost and approximate accuracy. However, there are many scenarios where the accuracy is subject to uncontrolled and large errors that originate from the underlying exchange correlation functional approximation. While it has recently become possible to incorporate highly accurate fully correlated ab initio wavefunction based methods in lieu of DFT for this part of the computation, these are prohibitively expensive as classical algorithms and are thus only applicable to very small system sizes. It is therefore desirable to develop an approach which retains the accuracy of ab initio wavefunction based methods but scales like DFT, and one way in which this can be realised is through more accurate exchange correlation potential approximations.

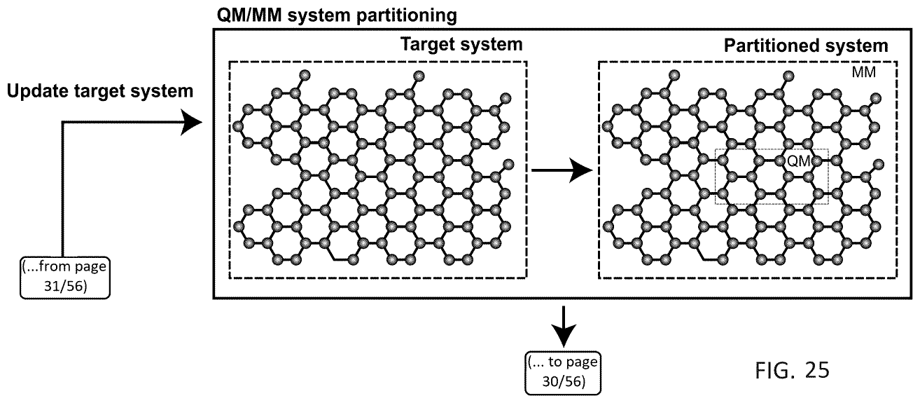

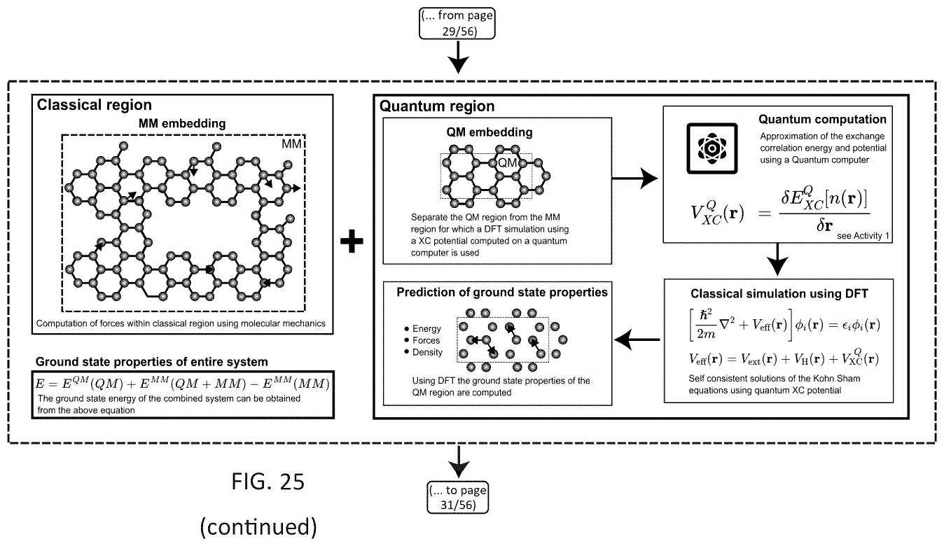

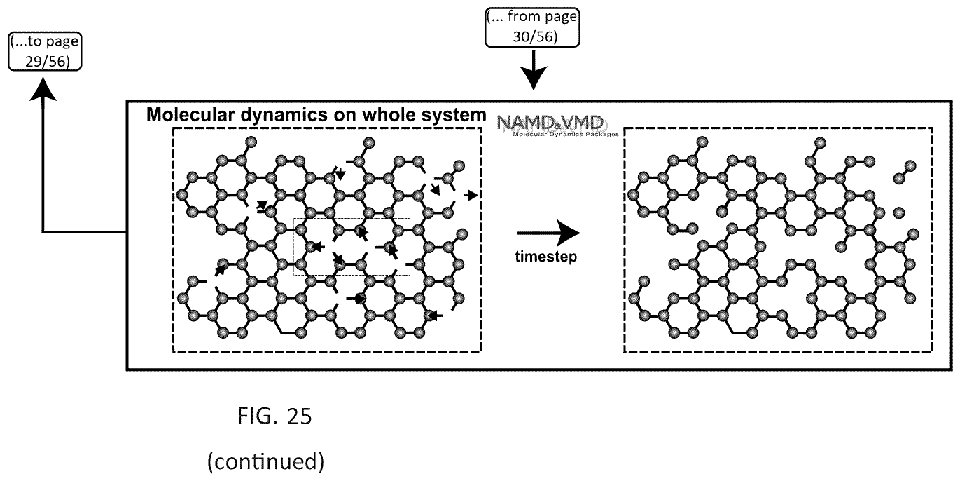

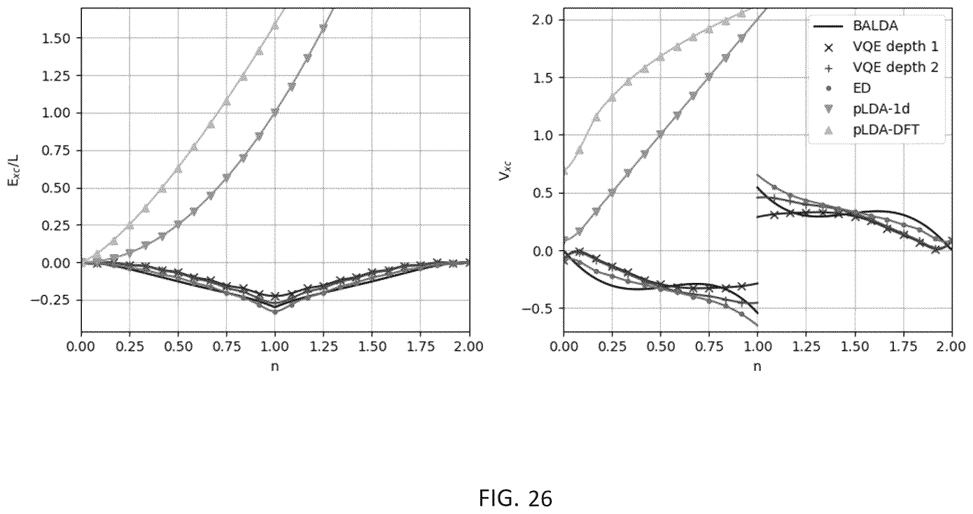

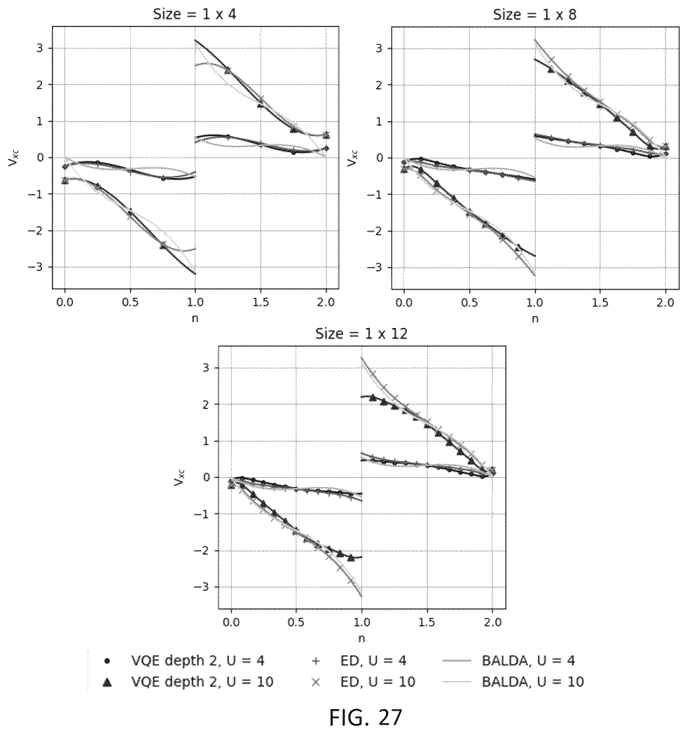

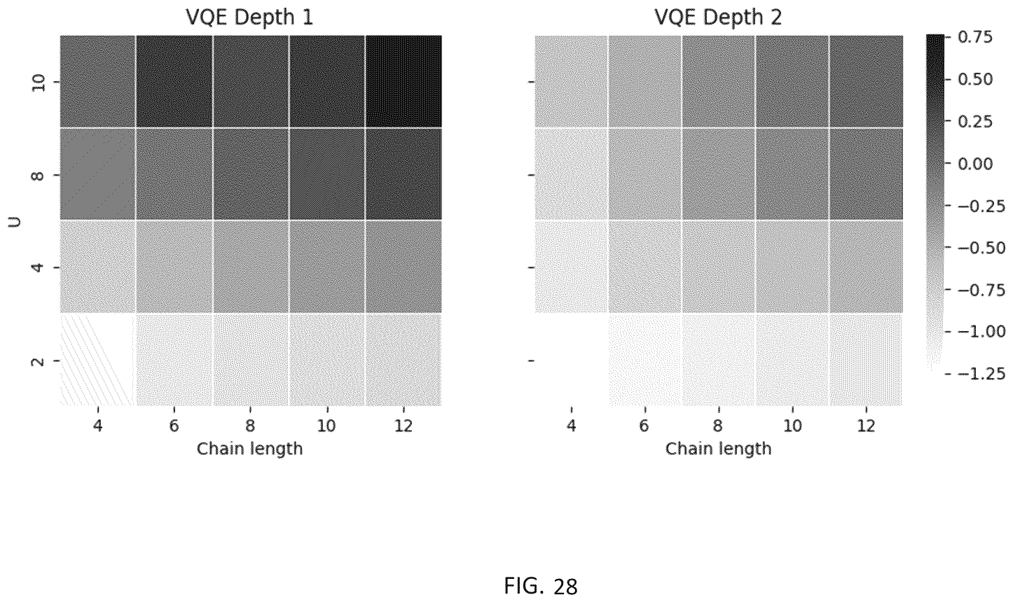

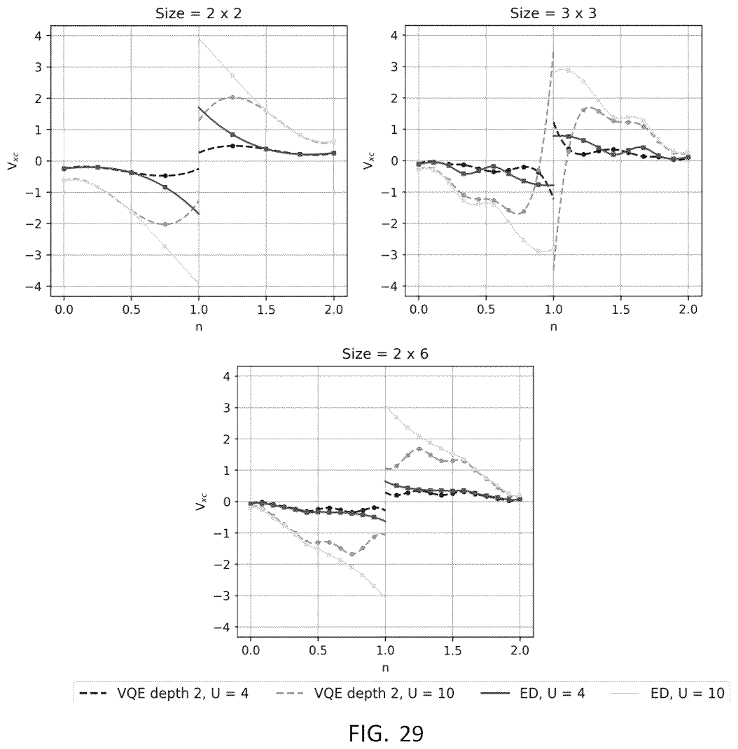

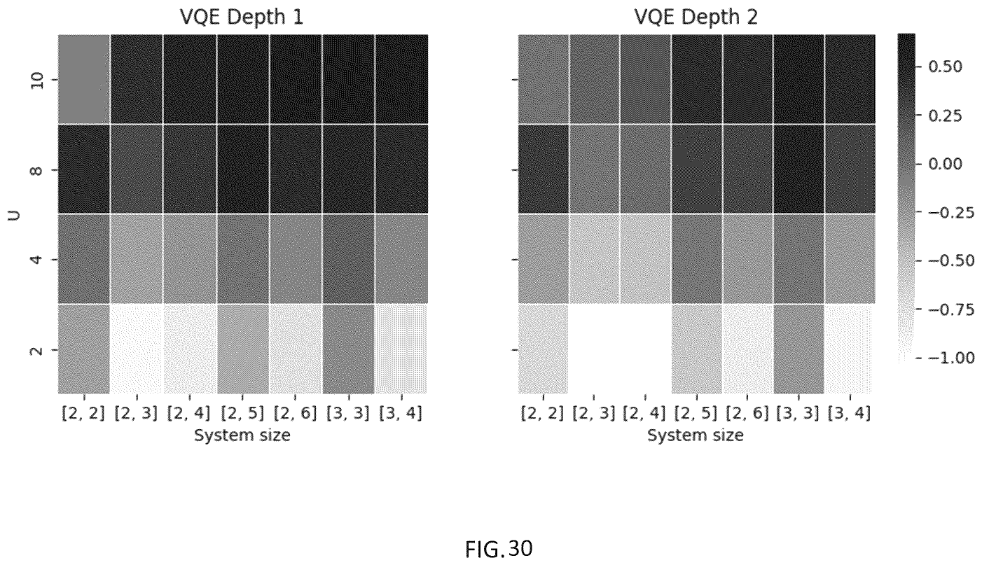

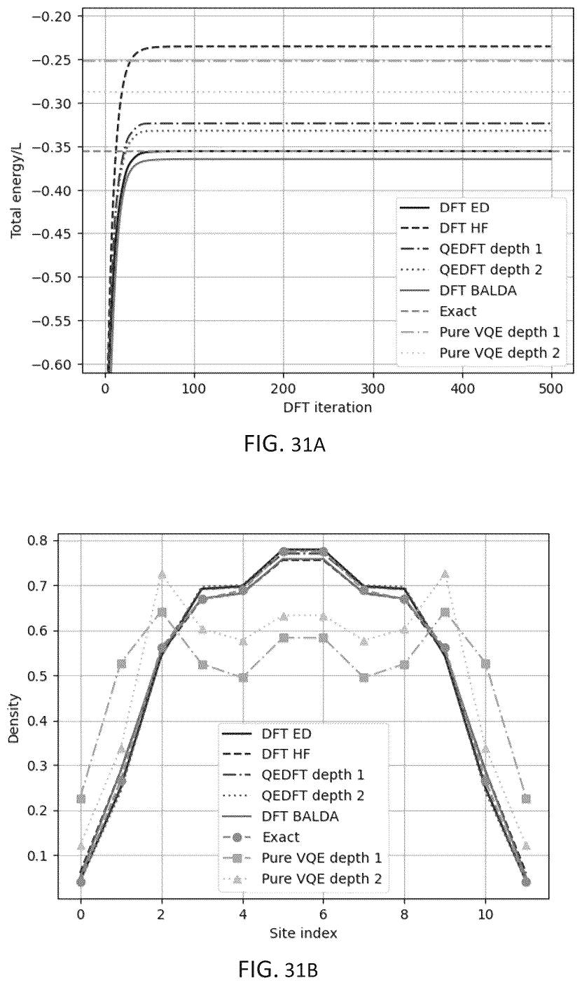

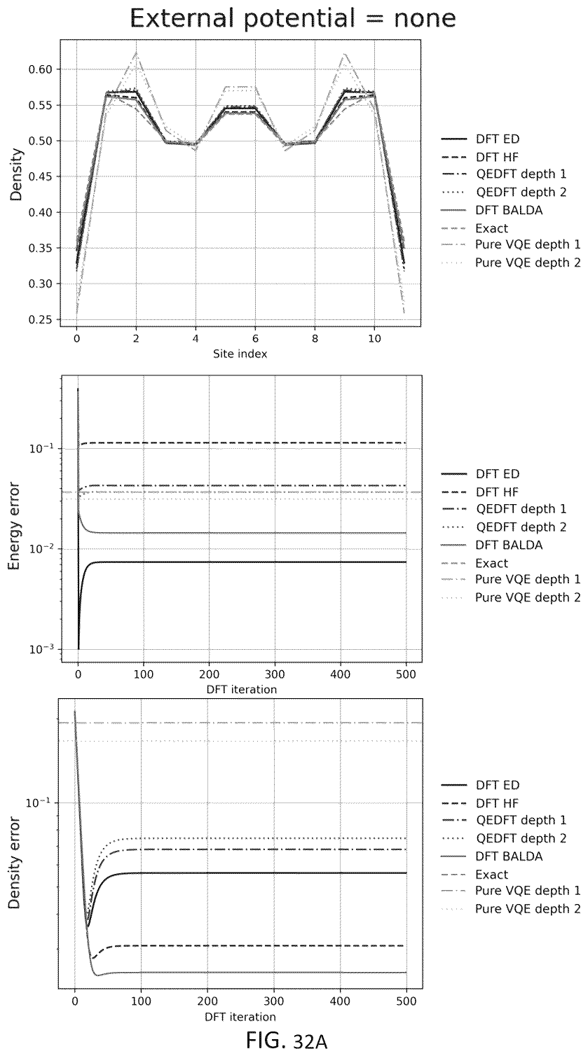

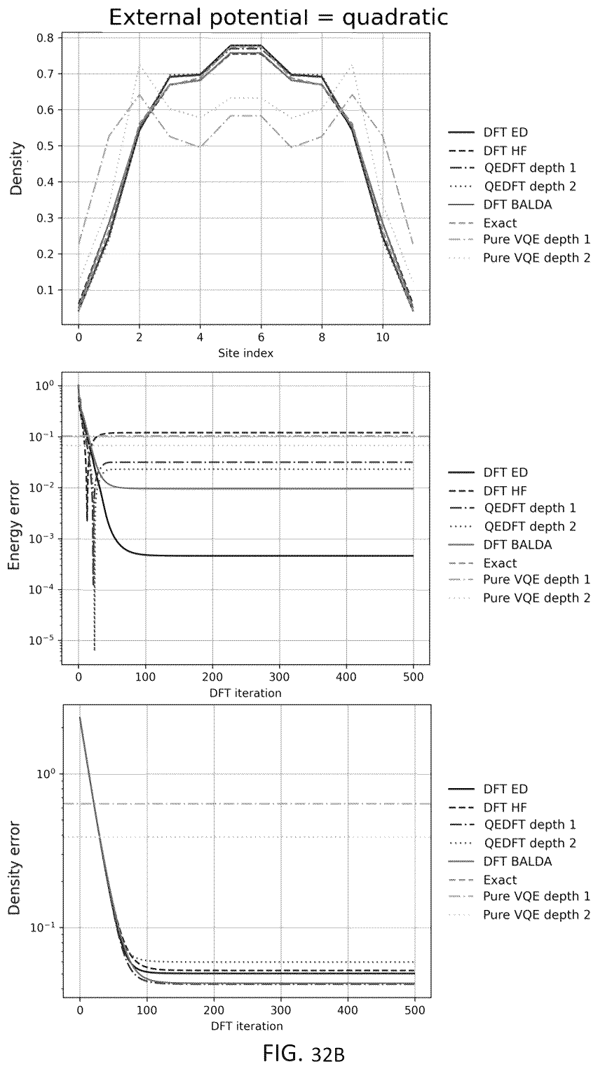

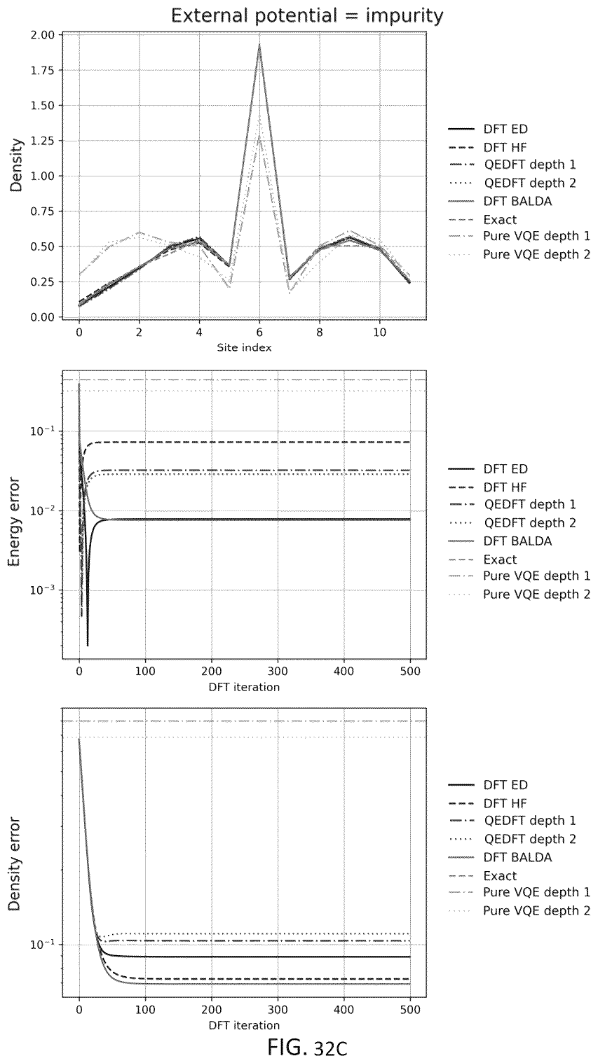

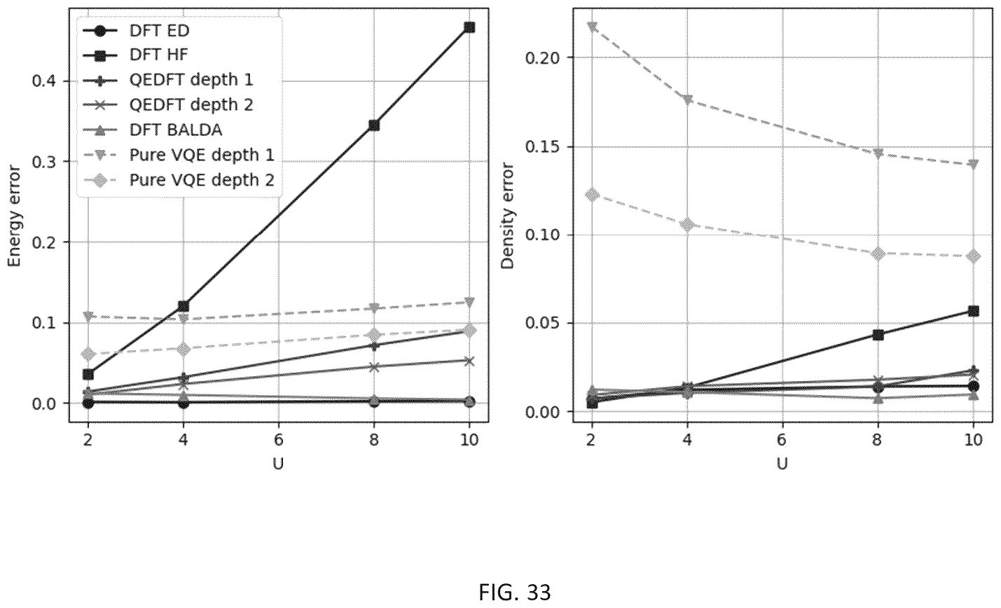

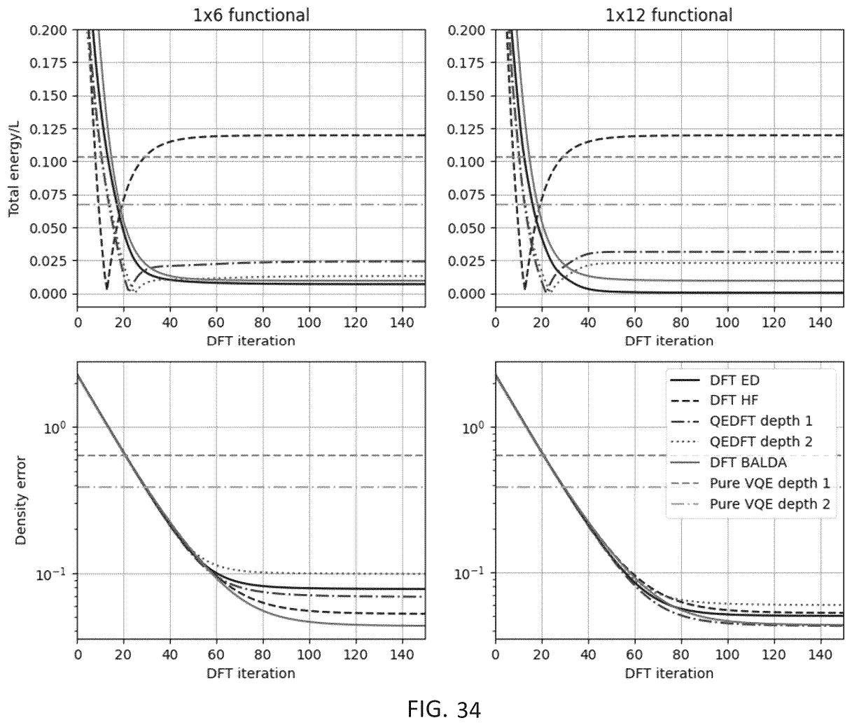

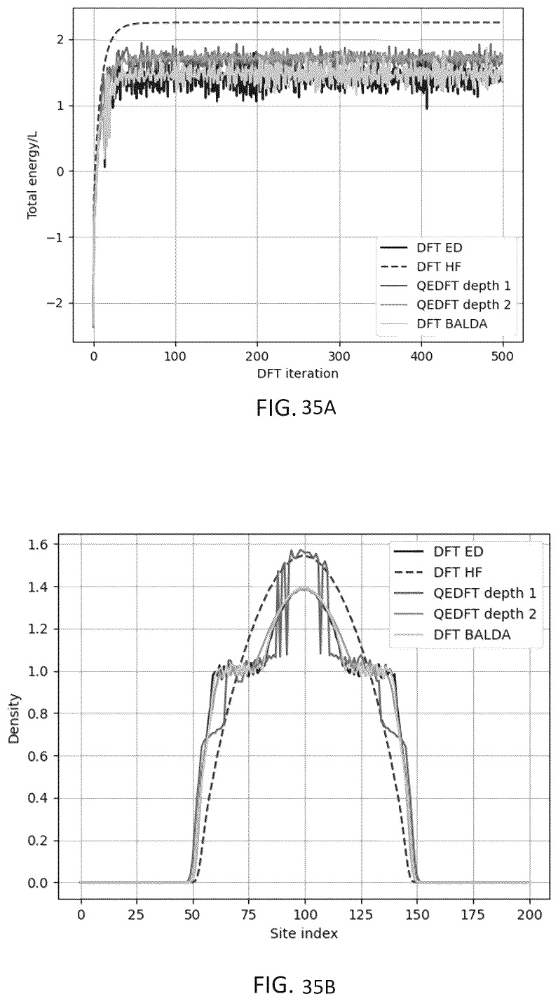

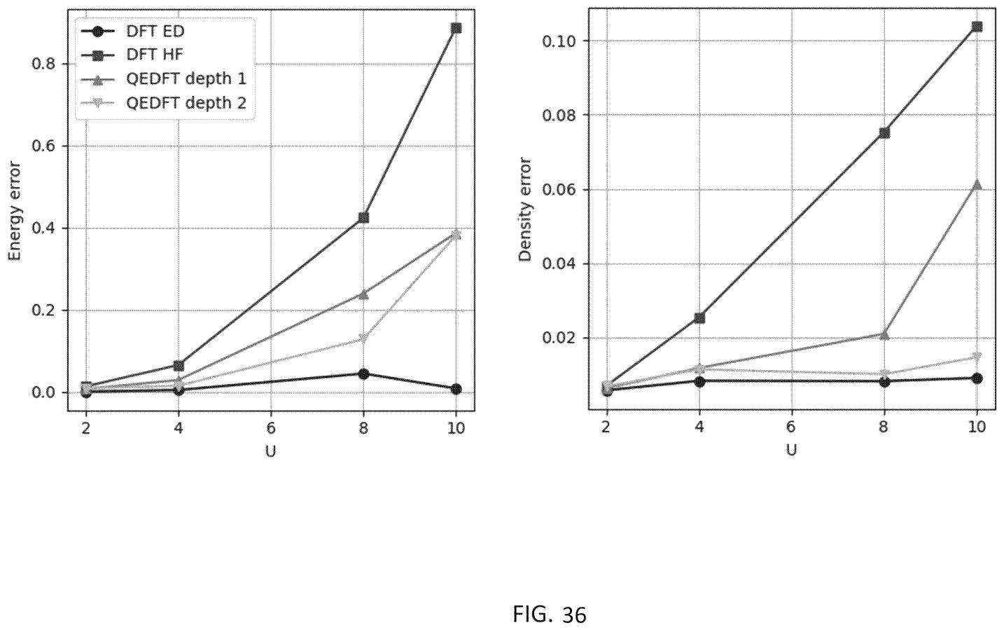

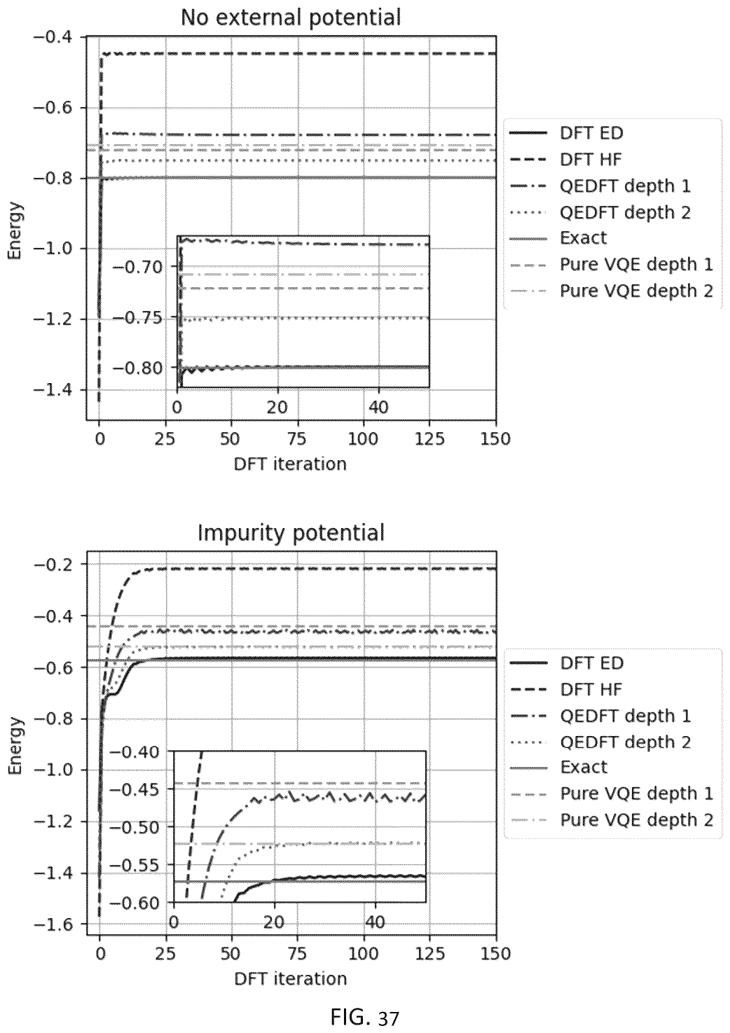

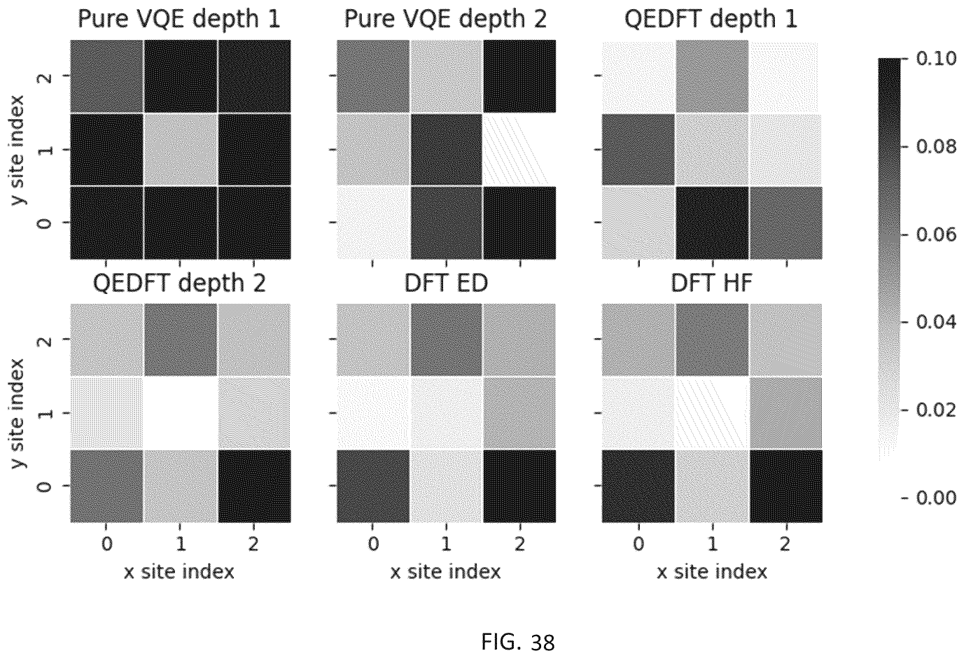

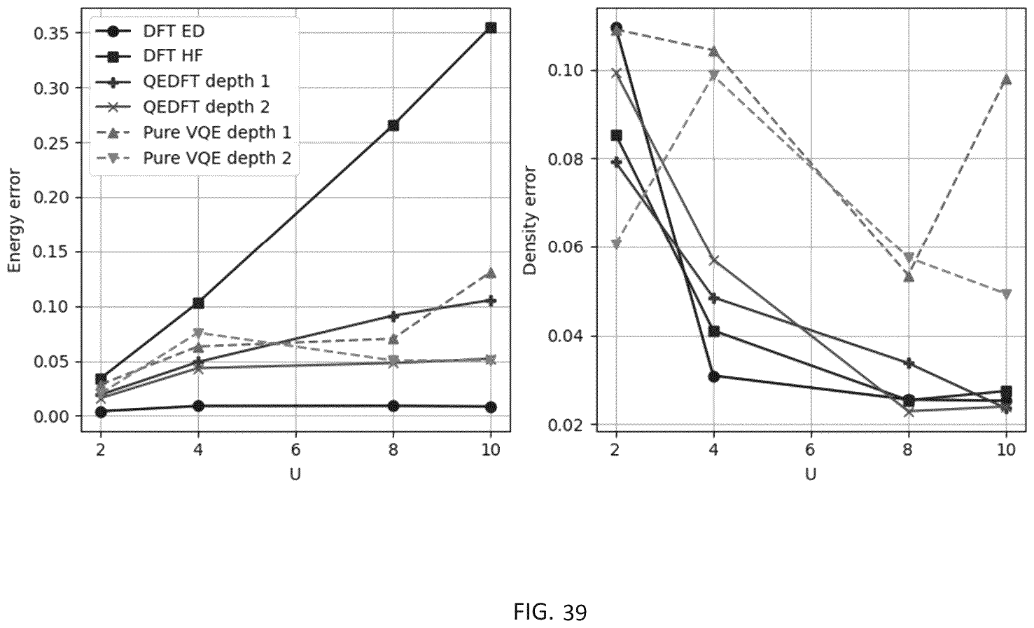

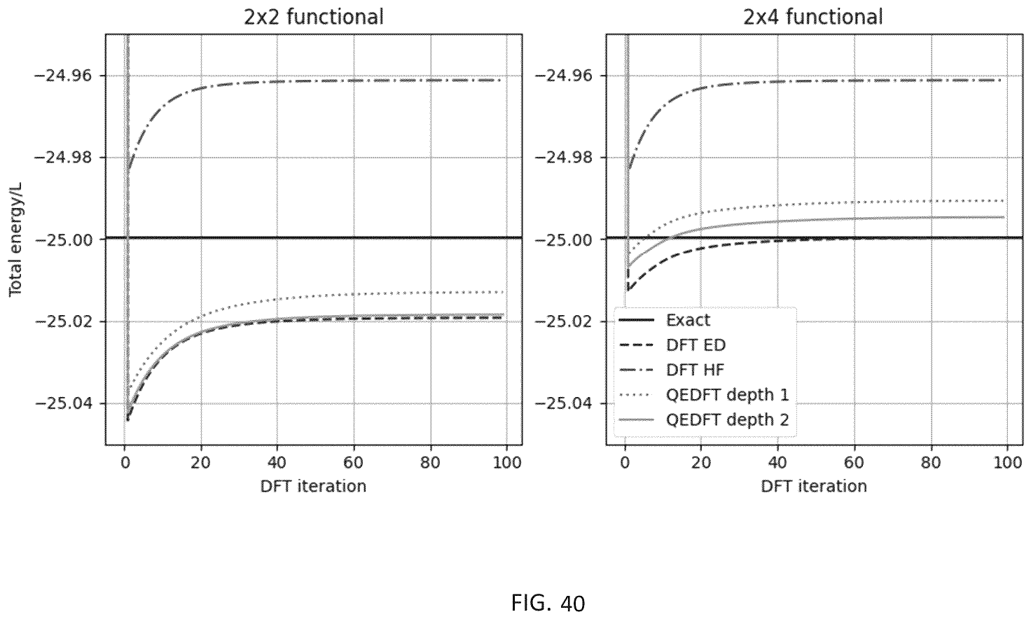

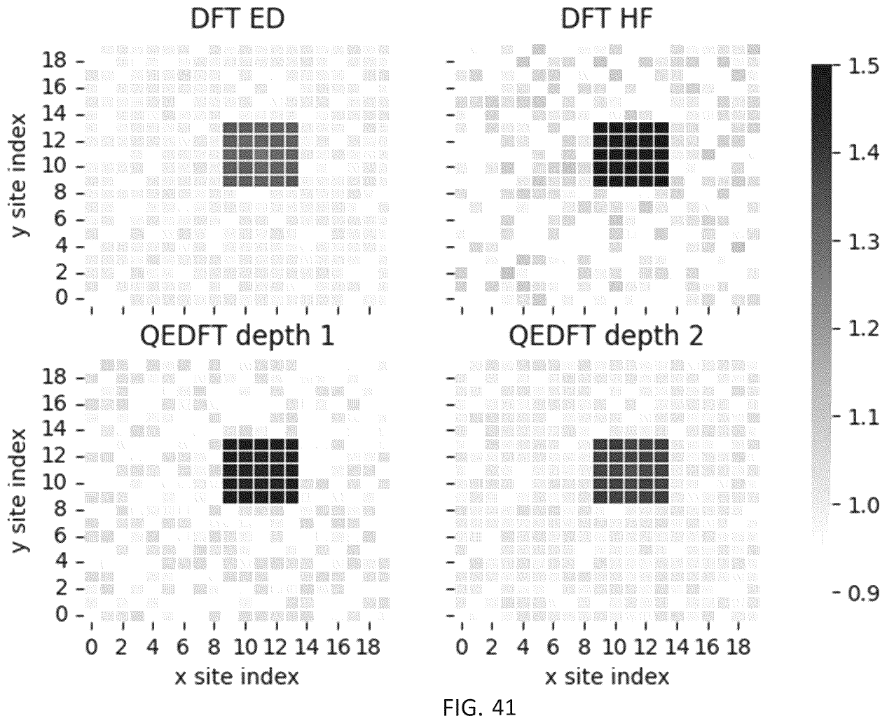

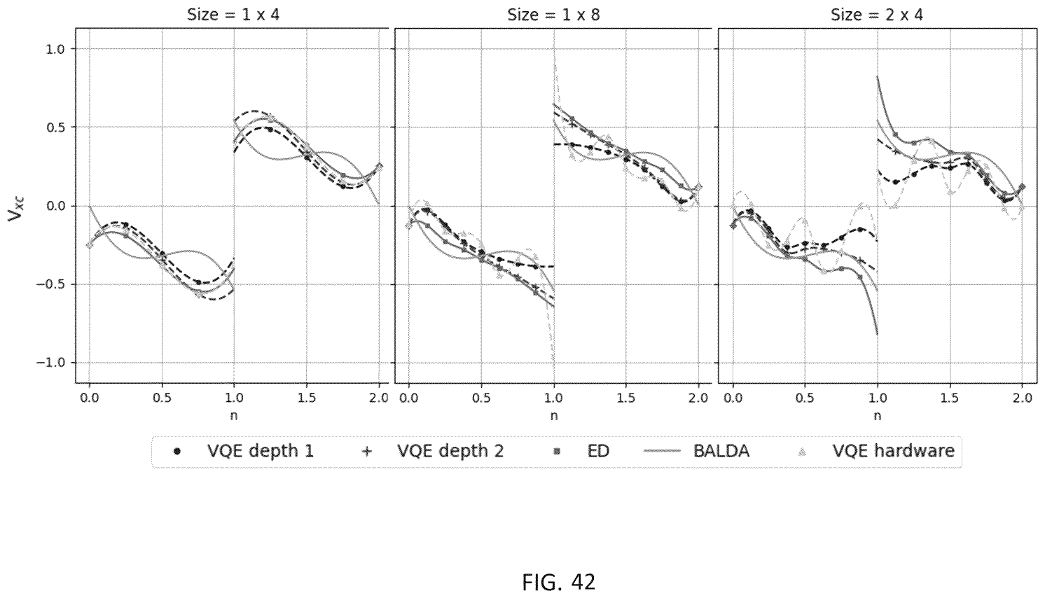

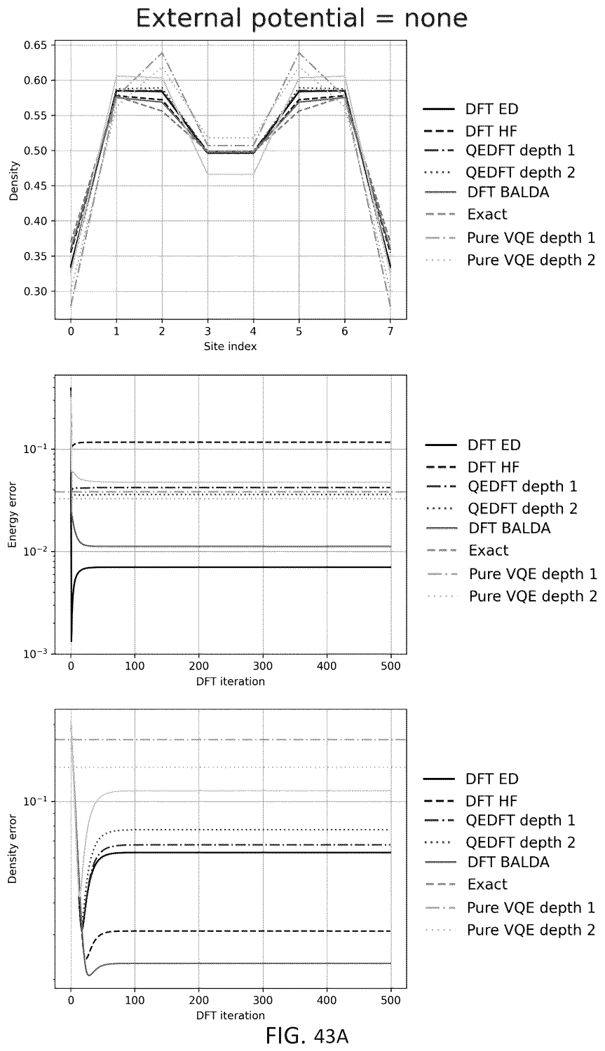

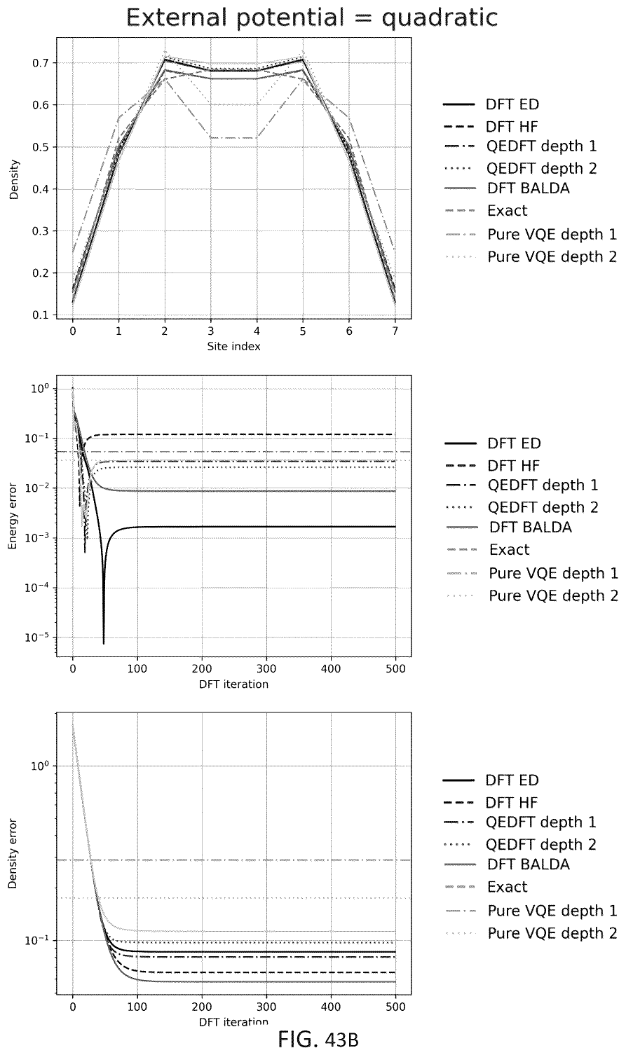

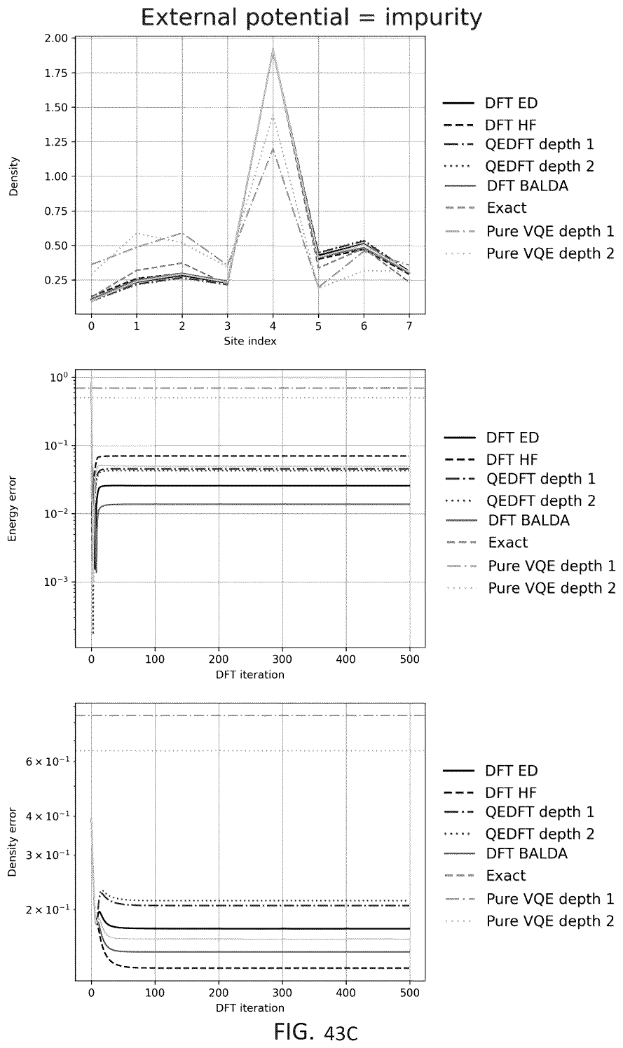

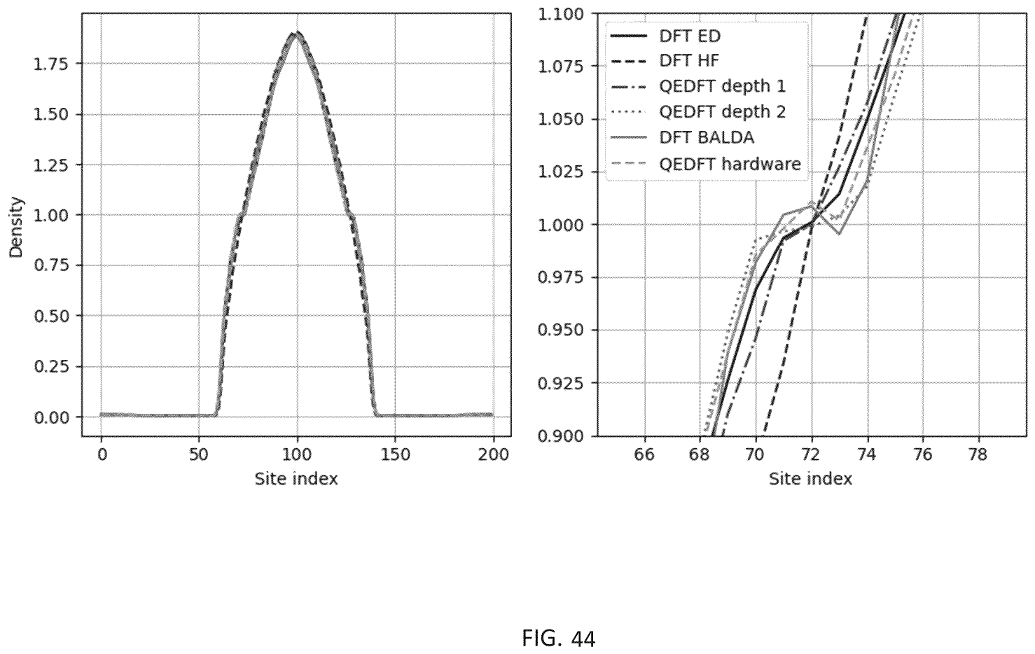

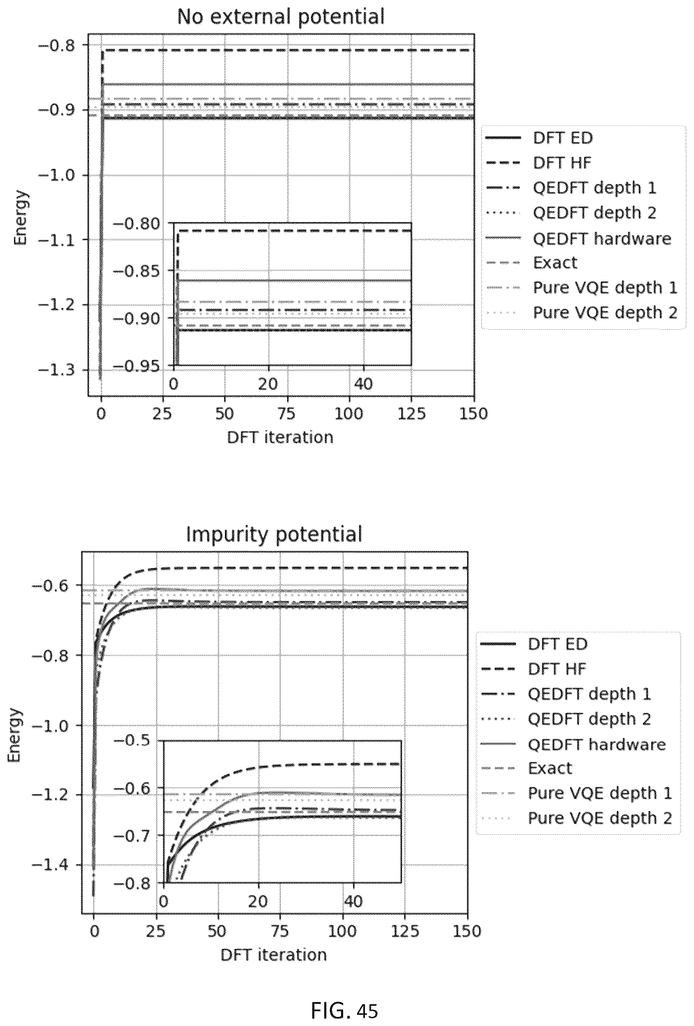

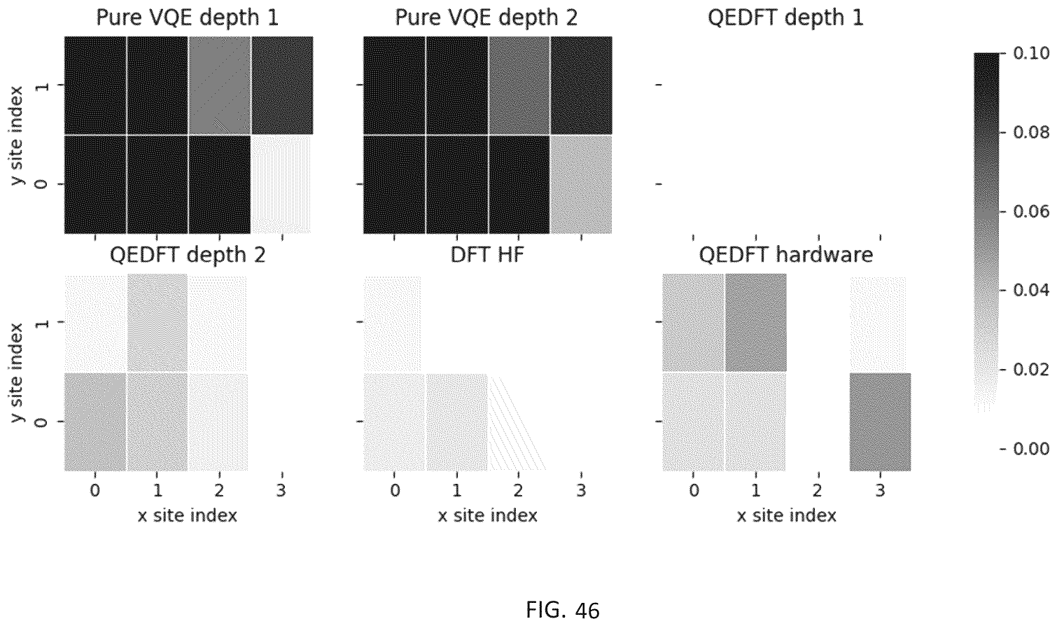

Therefore, we use the quantum algorithms developed in Activity 1 and show how they can be incorporated into the classical QM/MM framework described above, with a focus on leveraging specific features of near term quantum devices. Firstly, we explore in detail how the choice and size of the classical and quantum regions within the QM and MM regions of the simulation affect the quantum resource estimates of a black-box protocol for quantum enhanced single point hybrid QM/MM simulations. Moreover, we develop novel techniques that exploit the inherent advantages of using a quantum computer as a quantum solver. Ultimately, we use these advancements to assess the utility of these techniques for predicting a full reaction profile that incorporates dynamic correlation in a single point QM/MM calculation. The overall framework is illustrated in FIG. 25, charting the algorithmic workflow of how the data is passed back and forth between the hybrid quantum classical algorithm and highlighting where the intervention of the quantum computer occurs.

From this starting point, the work is well positioned to develop the theoretical evaluation of the quantum algorithm resource estimations, the novel techniques needed to exploit the quantum solvers for the QM/MM calculation, as well as establishing the computational interface between the classical and quantum computer, having developed a sizeable suite of proprietary software tools for classical-quantum algorithms in-house.

Activity 3a: Theoretical Evaluation and Resource Estimation of “Black Box” Use of Quantum Solver within QM/MM.

The black box use of a quantum solver within QM/MM can be viewed as taking an input molecular structure, as illustrated in FIG. 25, and performing molecular dynamics simulations that incorporate the dynamical correlation effects until the system is equilibrated. When considering the factors which affect the evaluation of this black box we focus on (1) the choice of the classical/quantum partition, i.e. the considered active space, and (2) the manner in which the system is combined after the separate MM (classical) and QM (quantum) simulations are complete. We achieve this by:
Theoretically develop the lower and upper bounds on the combined classical and quantum resource requirements (number of required qubits, program depth, classical program iterations) needed to efficiently represent the QM region inside the QM/MM framework as a quantum circuit using the approach shown in FIG. 25.
The recombination of the QM and MM regions can be achieved by incorporating the QM and MM interactive effects at different levels of theory. In FIG. 25 we highlight the naïve “subtractive” approach but we also explore the effect of the more advanced techniques, such as “additive” corrections have on the final quantum resources.
Perform a scaling study of how the number of active sites in the QM region affects the quantum resources using the QM/MM method with DFT functionals as opposed to the direct application of VQE on the QM active space for the computation of reaction profiles.
Incorporation of classical simulation codes (GROMACS) with proprietary software packages for quantum resource estimation, such as those discussed in our previous work on quantum resource estimation for materials simulation

### Activity 3b: Novel Techniques for QM/MM Integration Using the Features of Quantum Solvers.

As described in Activity 1, we use hybrid quantum-classical variational algorithms to construct approximations to the DFT XC functional. In particular, the performance of these solvers are sensitive to a number of factors, notably (1) the representation of the Hamiltonian for the QM region, (2) the fermionic encoding chosen to map the physical modes into qubits, and (3) the measurement strategy used to obtain ground state properties of the molecular system.

We develop novel techniques that consider each of these factors that aim to maximise the quantum resources available on near term devices. To achieve this, we:
Assess the effect of projector embedded methods, and further incorporate methods such as DMET, for the secondary truncation of the quantum active space within the QM region on quantum resource estimates. In this case, a further partitioning of the quantum subspace can be made at the QM level, such that the DFT computation is partitioned into “easy” and “difficult” computable regions, and the quantum computer only treats the “difficult” region, while the classical computer treats the “easy” region. Heretofore unpublished work by the inventors has indicated that these embedding schemes can significantly reduce the quantum resources for strongly correlated multiorbital Hubbard hamiltonians and we extend these techniques to the realistic example of Myotonic Dystrophy applications.
We explore the effect that different fermionic encoding schemes have on the description of the QM region, such as comparing the Jordan Wigner encoding to compact encoding schemes discussed by the inventors (see e.g. European patent application EP 23175487.0).
We create optimal measurement strategies that parallelise quantum computations in two ways. For example, the inventor's work on materials simulation highlights the importance of tailoring the measurement strategy for observables of choice, where parallelization across qubits can reduce the overall quantum runtime. Additionally, we assess the potential of using parallel-VQE, a recent parallel quantum solver framework proposed by the inventors, in improving the quality of results via executing parallel computations across the same chip, at the same time.

1. A method of performing improved Kohn-Sham Density Functional Theory, DFT, calculations, for simulation of a material, chemical, or biological system, comprising the steps of:
(i) constructing a model Hamiltonian of the system to be simulated;
(ii) inputting an electron density of the system to be simulated;
(iii) constructing a Kohn-Sham potential by:
(1) providing a second Hamiltonian based on the model Hamiltonian;
(2) calculating a ground state energy of the second Hamiltonian, as a function of electron density, using a quantum algorithm executed by a noisy intermediate-scale quantum, NISQ, processor;
(3) computing a quantum-obtained exchange correlation energy functional, as a function of electron density, from the NISQ processor-obtained ground state energy of step (2);
(4) obtaining a quantum-obtained exchange correlation potential functional by differentiation of the quantum-obtained exchange correlation energy functional from step (3);
(5) using the quantum-obtained exchange correlation potential functional from (4) to construct the Kohn-Sham potential; and
(iv) executing a quantum Kohn-Sham DFT algorithm iteration using the electron density from (ii) and the Kohn-Sham potential from (iii) to compute an updated electron density and an updated total energy of the system to be simulated.

2. The method according to claim 1, wherein when the model Hamiltonian provided in step (i) is inhomogeneous then the second Hamiltonian provided in step (1) is homogeneous, further wherein the model Hamiltonian has an external potential and the second Hamiltonian is based on the model Hamiltonian with the external potential removed.

3. The method according to claim 1, wherein calculating the ground state of the second Hamiltonian on an NISQ processor further comprises using a fermion-to-qubit encoding to encode qubits of the NISQ processor.

4. The method according to claim 3, wherein the fermion-to-qubit encoding is a Jordan Wigner encoding that maps fermionic creation and annihilation operators of the second Hamiltonian to Pauli strings comprising Pauli X, Y and Z operators.

5. The method according to claim 1, wherein calculating the ground state of the second Hamiltonian on an NISQ processor further comprises enacting qubit operations generated by unitary qubit operators on the qubits of the quantum information processor.

6. The method according to claim 1, wherein the model Hamiltonian is the Hubbard model with an external potential term, and the second Hamiltonian is the Hubbard model.

7. The method according to claim 1, wherein execution of the quantum algorithm by a noisy intermediate-scale quantum, NISQ, processor further comprises executing a Variational Quantum Eigensolver, VQE, algorithm using the NISQ processor.

8. The method according to claim 1, wherein obtaining a quantum-obtained exchange correlation potential functional in step (4) further comprises using a quantum kernel method for continuous representation of the exchange correlation energy functional as a function of electron density.

9. The method according to claim 1, wherein the material system to be simulated is a strongly correlated electron system.

10. The method according to claim 1, wherein the Kohn-Sham DFT algorithm is a Kohn-Sham Lattice Density Functional Theory, LDFT, algorithm.

11. The method according to claim 1, wherein the NISQ processor requires fewer qubits to calculate the ground state of the second Hamiltonian than the number of qubits required to calculate the ground state of the model Hamiltonian of the system to be simulated directly.

12. The method according to claim 1, wherein executing a quantum Kohn-Sham DFT algorithm iteration in step (iv) comprises the steps of:
(a) taking the electron density from (ii) and the Kohn-Sham potential from (iii) as an input to construct Kohn-Sham equations;
(b) solving the Kohn-Sham equations;
(c) providing the updated electron density, and the updated total energy of the system to be simulated, based on solutions of the Kohn-Sham equations in step (b); and
(d) checking if a convergence condition is met, further wherein the condition for convergence is evaluated based on the electron density input in step (ii) and the updated electron density.

13. The method according to claim 12, wherein evaluating the convergence condition in step (d) comprises calculating:
(α) a density difference metric from the electron density input in step (ii) and the updated electron density; and/or
(β) an energy difference metric from a total energy of the system to be simulated, based on the electron density from step (ii), and the updated total energy of the system to be simulated, And comparing the density difference metric from (α) and/or the density difference metric from (β) with a threshold condition for each difference metric.

14. The method according to claim 13, wherein further quantum Kohn-Sham DFT algorithm iterations, are performed by providing the updated electron density from (c) as the input in step (ii) if the convergence condition in (d) is not met such that steps (ii) to (iv) continue until the convergence condition for the electron density, and/or the convergence condition for the total energy of the system to be simulated, is met in step (d).

15. The method according to claim 13, wherein a further Kohn-Sham DFT iteration is performed in a classical manner, the classical Kohn-Sham DFT iteration comprising the steps (ii) to (iv), wherein step (ii) receives the updated electron density from (c) as an input and step (iii) replaces steps (1) to (5) by providing a classically obtained exchange correlation potential functional as an input to construct the Kohn-Sham potential.

16. The method according to claim 15, wherein further classical Kohn-Sham DFT iterations are performed until the convergence condition for the electron density, and/or the convergence condition for the total energy of the system to be simulated, is met in step (d).

17. The method according to claim 15, wherein further quantum or classical Kohn-Sham DFT algorithm iterations are performed, including at least one further quantum Kohn-Sham DFT iteration, until the convergence condition for the electron density, and/or the convergence condition for the total energy of the system to be simulated, is met in step (d).

18. The method according to claim 15, wherein further quantum Kohn-Sham DFT iterations are performed at periodic intervals with classical Kohn-Sham DFT algorithm iterations in between, until the convergence condition for the electron density, and/or the convergence condition for the total energy of the system to be simulated, is met in step (d).

19. A hybrid quantum-classical apparatus for simulation of a material, chemical, or biological system, by way of enacting the method of claim 1.

20. A non-transient computer readable medium comprising instructions which cause a computer to enact the method steps of claim 1.
